(19) United States
(12) Patent Application Publication
(10) Pub. No.: US 2024/0100121 A1
(43) Pub. Date: Mar. 28, 2024

(54) COMPOSITIONS OF A BETA-HAIRPIN PEPTIDOMIMETIC AND AEROSOL DOSAGE FORMS THEREOF

(71) Applicant: SPEXIS AG, Allschwil (CH)

(72) Inventors: Grégory UPERT, Kembs (FR); Mohammed BENGHEZAL, Villars-sur-Glâne (CH); Karen LE POUPON, Dietwiller (FR); Daniel OBRECHT, Bättwil (CH)

(21) Appl. No.: 18/253,852

(22) PCT Filed: Nov. 23, 2021
(86) PCT No.: PCT/EP2021/025457

§ 371 Date: May 22, 2023

(30) Foreign Application Priority Data
Nov. 23, 2020 (EP) 20020554.0
Apr. 8, 2021 (EP) 21020186.9

(51) Int. Cl. A61K 38/12; A61K 9/00; A61K 9/08; A61P 11/00; A61P 31/04; C07K 7/64

(52) U.S. Cl. CPC A61K 38/12; A61K 9/0078; A61K 9/08; A61P 11/00; A61P 31/04; C07K 7/64

(57) ABSTRACT

The invention relates to pharmaceutical aerosols comprising the active compound cyclo(-Thr-Trp-Ile-Dab-Orn-^{D}Dab-Dab-Trp-Dab-Dab-Ala-Ser-^{D}Pro-Pro), or any pharmaceutically acceptable salt thereof. It further relates to liquid compositions comprising the active compound cyclo(-Thr-Trp-Il-Dab-Orn-^{D}Dab-Dab-Trp-Dab-Dab-Ala-Ser-DPro-Pro), dilutions thereof, kits comprising the liquid compositions, pharmaceutical compositions and kits for preparing and administering such aerosols. The invention can be used for the prevention, management or treatment of diseases or conditions of the lungs such as cystic fibrosis (CF), non-cystic fibrosis bronchiectasis (NCFB), asthma, chronic obstructive pulmonary disease (COPD), ventilator-associated pneumonia (VAP), ventilator-associated bacterial pneumonia (VABP), hospital-acquired pneumonia (HAP), hospital-acquired bacterial pneumonia (HABP), or healthcare-associated pneumonia (HCAP).

### FIELD OF THE INVENTION

The invention relates to pharmaceutical aerosols comprising the active compound cyclo(-Thr-Trp-Ile-Dab-Orn-^{D}Dab-Dab-Trp-Dab-Dab-Ala-Ser-^{D}Pro-Pro), or any pharmaceutically acceptable salt thereof. It further relates to liquid compositions comprising the active compound cyclo(-Thr-Trp-Ile-Dab-Orn-^{D}Dab-Dab-Trp-Dab-Dab-Ala-Ser-^{D}Pro-Pro), dilutions thereof, kits comprising the liquid compositions, pharmaceutical compositions and kits for preparing and administering such aerosols. The invention can be used for the prevention, management or treatment of diseases or conditions of the lungs such as cystic fibrosis (CF), non-cystic fibrosis bronchiectasis (NCFB), asthma, chronic obstructive pulmonary disease (COPD), ventilator-associated pneumonia (VAP), ventilator-associated bacterial pneumonia (VABP), hospital-acquired pneumonia (HAP), hospital-acquired bacterial pneumonia (HABP), or healthcare-associated pneumonia (HCAP). Thus, the invention further relates to a pharmaceutical composition or a pharmaceutical aerosol comprising the active compound cyclo(-Thr-Trp-Ile-Dab-Orn-^{D}Dab-Dab-Trp-Dab-Dab-Ala-Ser-^{D}Pro-Pro) or any pharmaceutically acceptable salt thereof, for use in a method for the prevention, prophylaxis, management or treatment of diseases or conditions of the lungs in a subject, for use in a method for eradicating a biofilm in biofilm-associated diseases or conditions of the lungs in a subject, for use in a method for eradicating a biofilm in biofilm-associated diseases or conditions, or for use in a method for the prevention, management or treatment of biofilm formation in a subject.

### BACKGROUND OF THE INVENTION

The widespread use of antibiotics in the last half century is arguably the primary reason for increases in overall life-expectancy in developed nations. However, a consequence of this widespread use of antibiotics is the appearance of drug resistance, which negatively impacts the current treatment of bacterial diseases.

Although the pharmaceutical industry has responded to the concern over methicillin-resistant Staphylococcus aureus (MRSA) and other resistant Gram-positive pathogens, more recently, the increasing incidence of multi-drug resistant (MDR) Gram-negative pathogens such as Pseudomonas (P.) aeruginosa, has also become a major concern. P. aeruginosa, which has an intrinsic resistance to many antibiotics due to low cellular permeability and efficient drug efflux mechanisms, is particularly threatening in intensive care settings where long-term catheterization and/or ventilation are commonplace.

Bacterial lung infection is a major problem and may become life threatening for patient suffering from lung disorders, such as cystic fibrosis (CF), non-cystic fibrosis bronchiectasis (NCFB), asthma, chronic obstructive pulmonary disease (COPD), ventilator-associated pneumonia (VAP), ventilator-associated bacterial pneumonia (VABP), hospital-acquired pneumonia (HAP), hospital-acquired bacterial pneumonia (HABP), or healthcare-associated pneumonia (HCAP).

Specifically, cystic fibrosis (CF) is an autosomal recessive hereditary disease that affects approximately 30 000 people in the United States and approximately 40 000 in Europe. CF mutations occur in the gene encoding a chloride channel protein called the CF transmembrane conductance regulator (CFTR). Patients homozygous for defective CFTR genes typically suffer from chronic recurrent endobronchial infections (ultimately fatal) and sinusitis, as well as malabsorption due to pancreatic insufficiency, increased salt loss in sweat, obstructive hepatobiliary disease, and reduced fertility.

Pseudomonas aeruginosa (Pa) is the most significant pathogen in CF lung disease. Over 80% of CF patients eventually become colonized with Pa and the development of chronic infection of the lung with Pseudomonas aeruginosa is a typical feature of cystic fibrosis, which may induce further damage to the lung tissue and respiratory insufficiency, which is life threatening. One of the major factors contributing to the recalcitrant nature of these infections of the lung is the ability of Pseudomonas aeruginosa to form biofilms in these tissues.

A series of antibiotic compounds are disclosed in WO2007079605, respectively WO2007079597, which combine a high efficacy specifically against Pseudomonas aeruginosa with low hemotoxic effects. With the compounds described therein, a new strategy was introduced to stabilize β-hairpin conformations in backbone-cyclic cationic peptide mimetics exhibiting high selective antimicrobial activity. This involved transplanting the cationic and hydrophobic hairpin sequence onto a template, whose function is to restrain the peptide loop backbone into a hairpin geometry.

Template-bound hairpin mimetic peptides of this type have been also described in the literature (D. Obrecht, M. Altorfer, J. A. Robinson, Adv. Med. Chem. 1999, 4, 1-68; J. A. Robinson, Syn. Lett. 2000, 4, 429-441) and the ability to generate β-hairpin peptidomimetics using combinatorial and parallel synthesis methods has been established (L. Jiang, K. Moehle, B. Dhanapal, D. Obrecht, J. A. Robinson, Helv. Chim. Acta. 2000, 83, 3097-3112).

Routes of administration can be classified whether the effect of the drug is local (topical administration) or systemic (enteral or parenteral administration). The delivery of pharmaceuticals to the bronchi and lungs (pulmonary drug delivery) has been used for the local treatment of diseases and conditions of the respiratory system. However, the feasibility of inhalation as an alternate route of administration for treatment of systemic diseases utilising the large surface of the lungs for absorption has been demonstrated as well (J. S. Patton, P. R. Byron, Nat. Rev. Drug Discov. 2007, 6, 67-74; M. Hohenegger, Curr. Pharm. Des. 2010, 16, 2484-2492).

In particular, drug substances can be delivered to the respiratory system as aerosolized dry powders or aerosolized liquids, the latter being either solutions or dispersions, such as drug substance suspensions. Various devices have been developed to convert a solid or liquid composition into an aerosol to enable inhalation. For the conversion of aqueous-based drug substance solutions or suspensions into inhalable aerosols nebulizers are normally used. They are particularly useful for diseases that require high pulmonary doses, e.g. CF, and patients, e.g. children, who are unable to coordinate or achieve flow rates necessary for use of other inhalation devices (M. Knoch, M. Keller, Expert Opin. Drug Del. 2005, 2, 377-390).

Generally, the benefit of drug substance delivery via inhalation is that it can afford delivery of sufficient therapeutic dosages of the drug directly to the primary site of action, e.g. in case of respiratory diseases, while minimizing the risks of systemic toxicity. Additionally, suboptimal pharmacokinetics and/or pharmacodynamics associated with systemic drug exposure may be avoided. Furthermore, inhalation (at home) is a more convenient mode of administration than intravenous injection (medical ward).

In order to obtain an optimised formulation for aerosol administration, parameters like ratio dose/volume, osmolarity and pH need to be carefully considered. Formulation for aerosol delivery should contain the minimal yet efficacious amount of the active compound formulated in the smallest as possible volume of solution. In fact, the smallest the volume, the shortest the nebulisation time. A short nebulisation time, in turn, is an important determinant of patient compliance and within hospitals has implications for staff time (McCallion et al., Int. J. Pharm. 1996, 130, 1-11). It is well known that adverse reactions to inhalation therapy may be caused by hypo- or hyper-osmolarity of drug solutions. On the contrary, isotonic solutions remove the risk of paradoxical bronchoconstriction and cough (Mann et al., Br. Med. J. 1984, 289, 469). The osmolarity also affects the performances of the nebulisers in terms of output rate and particle size distribution.

An important requirement for an acceptable formulation is its adequate shelf-life suitable for commercial, distribution, storage and use. The stability of the active compound strictly depends on the pH. Therefore, the pH of its formulations need to be carefully adjusted in order to slow or prevent degradation products formation without the aid of preservatives and/or anti-oxidants; it would also be advantageous to adjust pH in such a way as to prevent as much as possible discoloration although the depth of colour is not a reliable indicator of the extent of oxidation. Formulations provided of adequate shelf-life under environmental storage conditions (room temperature and, at the occurrence, protected form light) would be particularly preferred, since the stability at room temperature of the preparations of the prior art are rather unsatisfactory.

Thus, there is a strong need for further optimized pharmaceutical compositions, aerosols and therapeutic kits which are suitable for the prevention, management or treatment of diseases or conditions of the lungs such as cystic fibrosis (CF), non-cystic fibrosis bronchiectasis (NCFB), asthma, chronic obstructive pulmonary disease (COPD), ventilator-associated pneumonia (VAP), ventilator-associated bacterial pneumonia (VABP), hospital-acquired pneumonia (HAP), hospital-acquired bacterial pneumonia (HABP), or healthcare-associated pneumonia (HCAP) improving the outcome of presently known therapies and/or overcoming the disadvantages of presently known therapies.

### SUMMARY OF THE INVENTION

The invention provides a pharmaceutical aerosol for pulmonary administration comprising a dispersed liquid phase and a continuous gas phase. The dispersed liquid phase comprises aqueous droplets comprising the active compound cyclo(-Thr-Trp-Ile-Dab-Orn-^{D}Dab-Dab-Trp-Dab-Dab-Ala-Ser-^{D}Pro-Pro); or any pharmaceutically acceptable salt thereof.

Thus in one aspect, the invention provides a liquid composition comprising from about 40 mg/mL to about 60 mg/mL of the active compound cyclo(-Thr-Trp-Ile-Dab-Orn-^{D}Dab-Dab-Trp-Dab-Dab-Ala-Ser-^{D}Pro-Pro), wherein the composition has a pH from about 3.5 to about 4.5.

In a further aspect, the invention provides a kit comprising: (a) first kit component comprising a liquid composition comprising from about 40 mg/mL to about 60 mg/mL of the active compound cyclo(-Thr-Trp-Ile-Dab-Orn-^{D}Dab-Dab-Trp-Dab-Dab-Ala-Ser-^{D}Pro-Pro), wherein the composition has a pH from about 3.5 to about 4.5; and (b) a second kit component comprising a diluent.

In a further aspect, the invention provides a dilution of a liquid composition comprising from about 40 mg/mL to about 60 mg/mL of the active compound cyclo(-Thr-Trp-Ile-Dab-Orn-^{D}Dab-Dab-Trp-Dab-Dab-Ala-Ser-^{D}Pro-Pro), wherein the composition has a pH from about 3.5 to about 4.5, wherein the dilution comprises from about 20 mg/mL to about 30 mg/mL of the active compound cyclo(-Thr-Trp-Ile-Dab-Orn-^{D}Dab-Dab-Trp-Dab-Dab-Ala-Ser-^{D}Pro-Pro), and wherein the dilution has a pH from about 6.5 to about 7.5.

In a further aspect, the invention provides a pharmaceutical composition comprising the active compound cyclo(-Thr-Trp-Ile-Dab-Orn-^{D}Dab-Dab-Trp-Dab-Dab-Ala-Ser-^{D}Pro-Pro); or any pharmaceutically acceptable salt thereof; and optionally one or more pharmaceutically acceptable diluents, excipients or carriers; wherein the pharmaceutical composition comprises from about 1.5 mg/mL to about 25 mg/mL of the active compound cyclo(-Thr-Trp-Ile-Dab-Orn-^{D}Dab-Dab-Trp-Dab-Dab-Ala-Ser-^{D}Pro-Pro).

In a further aspect, the invention provides a pharmaceutical composition comprising the active compound cyclo(-Thr-Trp-Ile-Dab-Orn-^{D}Dab-Dab-Trp-Dab-Dab-Ala-Ser-^{D}Pro-Pro); or any pharmaceutically acceptable salt thereof; and optionally one or more pharmaceutically acceptable diluents, excipients or carriers; wherein the pharmaceutical composition comprises from about 1.5 mg/mL to about 25 mg/mL of the active compound cyclo(-Thr-Trp-Ile-Dab-Orn-^{D}Dab-Dab-Trp-Dab-Dab-Ala-Ser-^{D}Pro-Pro) for use in a method for the prevention, management or treatment of diseases or conditions of the lungs in a subject.

In a further aspect, the invention provides a pharmaceutical aerosol for pulmonary administration comprising a dispersed liquid phase and a continuous gas phase, wherein the dispersed liquid phase
(a) comprises aqueous droplets comprising the active compound cyclo(-Thr-Trp-Ile-Dab-Orn-^{D}Dab-Dab-Trp-Dab-Dab-Ala-Ser-^{D}Pro-Pro);
or any pharmaceutically acceptable salt thereof;
(b) has a mass median aerodynamic diameter from about 1.5 μm to about 5 μm; and
(c) has a droplet size distribution having a geometrical standard deviation from about 1.2 to about 1.8.

In a further aspect, the invention provides a pharmaceutical aerosol for pulmonary administration comprising a dispersed liquid phase and a continuous gas phase, wherein the dispersed liquid phase
(a) comprises aqueous droplets comprising the active compound cyclo(-Thr-Trp-Ile-Dab-Orn-^{D}Dab-Dab-Trp-Dab-Dab-Ala-Ser-^{D}Pro-Pro);
or any pharmaceutically acceptable salt thereof;
(b) has a mass median aerodynamic diameter from about 1.5 μm to about 5 μm; and
(c) has a droplet size distribution having a geometrical standard deviation from about 1.2 to about 1.8;
for use in a method for the prevention, management or treatment of diseases or conditions of the lungs in a subject.

In a further aspect, the invention provides a kit for the preparation and delivery of a pharmaceutical aerosol for pulmonary administration comprising a dispersed liquid phase and a continuous gas phase, wherein the dispersed liquid phase
(a) comprises aqueous droplets comprising the active compound cyclo(-Thr-Trp-Ile-Dab-Orn-^{D}Dab-Dab-Trp-Dab-Dab-Ala-Ser-^{D}Pro-Pro);
or any pharmaceutically acceptable salt thereof;
(b) has a mass median aerodynamic diameter from about 1.5 μm to about 5 μm; and
(c) has a droplet size distribution having a geometrical standard deviation from about 1.2 to about 1.8;
and wherein the kit comprises a nebulizer and a liquid composition comprising a concentration within a range from about 1.5 mg/mL to about 25 mg/mL of the active compound.

In a further aspect, the invention provides a pharmaceutical composition comprising the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof;
and optionally one or more pharmaceutically acceptable diluents, excipients or carriers; for use in a method for the prevention, management or treatment of biofilm-associated diseases or conditions of the lungs in a subject.

In a further aspect, the invention provides a pharmaceutical composition comprising the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof;
and optionally one or more pharmaceutically acceptable diluents, excipients or carriers; for use in a method for eradicating a biofilm in biofilm-associated diseases or conditions of the lungs in a subject.

In a further aspect, the invention provides a pharmaceutical composition comprising the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof;
and optionally one or more pharmaceutically acceptable diluents, excipients or carriers; for use in a method for eradicating a biofilm in biofilm-associated diseases or conditions in a subject.

In a further aspect, the invention provides a pharmaceutical composition comprising the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof;
and optionally one or more pharmaceutically acceptable diluents, excipients or carriers; for use in a method for the prevention, management or treatment of biofilm formation in a subject.

The liquid compositions and dilutions thereof comprising the active compound cyclo(-Thr-Trp-Ile-Dab-Orn-^{D}Dab-Dab-Trp-Dab-Dab-Ala-Ser-^{D}Pro-Pro) can be used to prepare the pharmaceutical compositions and the liquid phase of the pharmaceutical aerosol of the present invention and provide an excellent stability profile and allow to prepare pharmaceutical aerosols with an osmolality required for administration by nebulization.

### BRIEF DESCRIPTION OF THE FIGURES

FIG. 1 shows the stability of the solutions described in Examples 1-3 over time at 5° C.

FIG. 2 shows the stability of the solutions described in Examples 1-3 over time at 25° C.

FIG. 3 shows a schematic representation of the applied methodology when performing the biofilm assays by the Calgary device (closed system) as described in Example 17.

FIG. 4 shows the results of the biofilm assay performed by the BioFlux microfluidic open system as described in Example 17.

### DETAILED DESCRIPTION OF THE INVENTION

In case of conflict, the present specification including the definitions therein will prevail. Unless defined otherwise, all technical and scientific terms used herein will have the same meaning as commonly understood by one of skill in the art to which the subject-matter herein belongs. As used herein, the following definitions are supplied in order to facilitate the understanding of the present invention.

As used herein, the term “comprise/comprising” is generally used in the sense of include/including, i.e. permitting the presence of one or more features or components. The terms “comprise” and “comprising” also encompass the more restricted terms “consist” and “consisting”.

As used herein, the singular form “a”, “an” and “the” include plural references unless the context clearly dictates otherwise.

As used herein, the term “administering” in relation to an active compound, e.g., cyclo(-Thr-Trp-Ile-Dab-Orn-^{D}Dab-Dab-Trp-Dab-Dab-Ala-Ser-^{D}Pro-Pro-), is used to refer to delivery of that compound by any route of delivery.

As used herein, the term “about” in relation to a numerical value x means+/−10%.

As used herein, the word “substantially” does not exclude “completely,” e.g., a composition which is “substantially free” from Y may be completely free from Y.

As used herein, the terms “patient” or “subject” is well-recognized in the art and can be used interchangeably and is preferably a human subject. They can refer to a mammal, including dog, cat, rat, mouse, monkey, cow, horse, goat, sheep, pig, camel, and, most preferably, a human. In some embodiments, the subject is a subject in need of treatment or a subject with a disease or disorder affecting the lungs. However, in other embodiments, the subject can be a normal subject or a subject who has already undergone a treatment a disease or disorder affecting the lungs. The term does not denote a particular age or sex. Thus, all male or female adults, adolescents, children and newborn subjects, are intended to be covered.

The term “pharmaceutically acceptable diluent, excipient or carrier” as used herein refers to a carrier or excipient or diluent that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

The term “biofilm” as used herein refers to an aggregate of bacterial microorganisms, in particular Pseudomonas aeruginosa, in which bacterial cells adhere to each other and/or to a surface. These adherent cells are often covered with a matrix of extracellular polymeric substance (EPS), which is produced by the cells. Biofilm EPS has been characterized as composed of extracellular DNA, proteins, and polysaccharides. Such biofilms may form on any living or non-living surfaces, in particular both on solid surfaces as colonies and/or on liquid surfaces as pellicles. Microbial cells growing in a biofilm are physiologically distinct from planktonic cells of the same organism. In certain aspects of the invention, bacterial formations within the gastrointestinal tract of a subject are defined as biofilms if such formations are of a minimal size. In particular, exemplified biofilms were characterized as a massive bacterial invasion (>10⁹ bacteria/ml) of the mucus layer spanning at least a linear distance of 200 μm of the epithelial surface; however, as described in greater detail below, a range of bacterial density and/or size cutoffs may be selected as defining a biofilm within a subject.

The term “biofilm-associated disease” as used herein refers to a disease or condition as described herein which is related to or affected by the formation of a biofilm of bacterial cells, in particular caused by Pseudomonas aeruginosa.

The phrase “eradicating a biofilm” as used herein refers to the capacity of a substance to effect a disruption and/or the eradication of an established and/or matured biofilm of bacterial cells, in particular Pseudomonas aeruginosa; and/or to effect a reduction in the rate of buildup of a biofilm of bacterial cells, in particular Pseudomonas aeruginosa, on a surface (e.g., within the lungs of a subject).

In a first aspect, the present invention provides a liquid composition comprising from about 40 mg/mL to about 60 mg/mL of the active compound cyclo(-Thr-Trp-Ile-Dab-Orn-^{D}Dab-Dab-Trp-Dab-Dab-Ala-Ser-^{D}Pro-Pro-), wherein the composition has a pH from about 3.5 to about 4.5.

In the active compound cyclo(-Thr-Trp-Ile-Dab-Orn-^{D}Dab-Dab-Trp-Dab-Dab-Ala-Ser-^{D}Pro-Pro)
Dab is (S)-2,4-diaminobutanoic acid;
^{D}Dab is (R)-2,4-diaminobutanoic acid;
Orn is (S)-2,5-diaminopentanoic acid;
   and all other amino acid residues are L-amino acid residues, if not explicitly designated as D-amino acid residues, following standard IUPAC nomenclature.

For avoidance of doubt, hereinafter follows a list of abbreviations, corresponding to generally adopted usual practice, of amino acids which, or the residues of which, are suitable for the purposes of the present invention and referred to in this document.

The descriptors L respectively D, e.g. in ^{D}Pro, refer to the stereochemistry at the α-position of the α-amino acid and are used according the Fischer-Rosanoff convention of the IUPAC.

| | | |
|---|---|---|
| Ala | L-Alanine | (S)-2-aminopropanoic acid |
| Ile | L-Isoleucine | (2S,3S)-2-amino-3-methylpentanoic acid |
| Orn | L-Ornithine | (S)-2,5-diaminopentanoic acid |
| Pro | L-Proline | (S)-2-pyrrolidinecarboxylic acid |
| ^{D}Pro | D-Proline | (R)-2-pyrrolidinecarboxylic acid |
| Ser | L-Serine | (S)-2-amino-3-hydroxypropanoic acid |
| Thr | L-Threonine | (2S,3R)-2-amino-3-hydroxybutanoic acid |
| Trp | L-Tryptophan | (S)-2-Amino-3-(1H-indol-3-yl)propanoic acid |
| Dab | | (S)-2,4-diaminobutanoic acid |
| ^{D}Dab | | (R)-2,4-diaminobutanoic acid; |

As used herein in the description and the claims the active compound cyclo(-Thr-Trp-Ile-Dab-Orn-^{D}Dab-Dab-Trp-Dab-Dab-Ala-Ser-^{D}Pro-Pro) should be understood as net peptide. Solvates as well as salts are categories of forms in which the active compound cyclo(-Thr-Trp-Ile-Dab-Orn-^{D}Dab-Dab-Trp-Dab-Dab-Ala-Ser-^{D}Pro-Pro) may be used as an active ingredient in a pharmaceutical composition.

Salts are neutral compounds composed of ions, i.e. cations and anions. If the active compound can act like an acid, potentially useful salts may be formed with inorganic cations, such as sodium, potassium, calcium, magnesium and/or ammonium, or with organic cations, such as those derived from arginine, lysine, glycine, and/or ethylenediamine. If the active compound (or parts thereof) can act like a base, as for example the residue of Orn being one of the amino acid residues of cyclo(-Thr-Trp-Ile-Dab-Orn-^{D}Dab-Dab-Trp-Dab-Dab-Ala-Ser-^{D}Pro-Pro), then potentially useful salts may be formed with inorganic or organic anions, such as chloride, bromide, iodide, phosphate (mono- or dibasic), sulfate, nitrate, acetate, trifluoroacetate, propionate, butyrate, maleate, fumarate, methanesulfonate, ethanesulfonate, 2-hydroxy-ethylsulfonate, n-propylsulfonate, isopropylsulfonate, lactate, malate, and/or citrate.

The term pharmaceutically acceptable salt or pharmaceutical salt is used to refer to an ionisable drug or active compound that has been combined with a counter ion to form a neutral complex. Converting a drug or active compound into a salt through this process can, for example, increase its chemical stability, render the complex easier to administer and/or allow manipulation of the agent's pharmacokinetic profile. In a preferred embodiment of the invention, the counter ion of the active compound is acetate.

In a preferred embodiment of the invention, the liquid composition is an aqueous composition. In a further preferred embodiment of the invention the acetate salt of the active compound cyclo(-Thr-Trp-Ile-Dab-Orn-^{D}Dab-Dab-Trp-Dab-Dab-Ala-Ser-^{D}Pro-Pro-) is dissolved in the aqueous composition.

In a preferred embodiment of the invention the liquid composition or the aqueous composition has a pH of about 4.0.

In a preferred embodiment of the invention the liquid composition or the aqueous composition comprises about 50 mg/mL of the active compound cyclo(-Thr-Trp-Ile-Dab-Orn-^{D}Dab-Dab-Trp-Dab-Dab-Ala-Ser-^{D}Pro-Pro-).

In a further aspect, the present invention provides a kit comprising:
(a) a first kit component comprising a liquid composition comprising from about 40 mg/mL to about 60 mg/mL of the active compound cyclo(-Thr-Trp-Ile-Dab-Orn-^{D}Dab-Dab-Trp-Dab-Dab-Ala-Ser-^{D}Pro-Pro-), wherein the composition has a pH from about 3.5 to about 4.5; and
(b) a second kit component comprising a diluent.

In a preferred embodiment of the invention the diluent of the second kit component is an aqueous diluent. In a further preferred embodiment the aqueous diluent comprises at least one buffering agent. In a further preferred embodiment the diluent of the second kit component is an aqueous diluent comprising at least one buffering agent wherein the at least one buffering agent is adapted to maintain a pH from about 6.5 to about 7.5, preferably a pH of about 7.0, upon combining the first and the second kit components. In a further preferred embodiment the diluent of the second kit component is an aqueous diluent comprising a phosphate buffer, preferably a 200 mM phosphate buffer, and has a pH from about 11.0 to about 13.5, preferably from about 13.0 to about 13.5, more preferably about 13.1. Usually the aqueous diluent is prepared by mixing a phosphate buffer, preferably a 200 mM phosphate buffer with pH 7 with NaOH 1 N and water for injection.

In a further aspect, the present invention provides a dilution of a liquid composition comprising from about 40 mg/mL to about 60 mg/mL of the active compound cyclo(-Thr-Trp-Ile-Dab-Orn-^{D}Dab-Dab-Trp-Dab-Dab-Ala-Ser-^{D}Pro-Pro-), wherein the liquid composition has a pH from about 3.5 to about 4.5, wherein the dilution comprises from about 20 mg/mL to about 30 mg/mL of the active compound cyclo(-Thr-Trp-Ile-Dab-Orn-^{D}Dab-Dab-Trp-Dab-Dab-Ala-Ser-^{D}Pro-Pro-), and has a pH from about 6.5 to about 7.5, preferably a pH of about 7.0 and optionally a osmolality from about 150 to about 500 mOsm/kg, preferably from about 200 to about 400 mOsm/kg.

In a preferred embodiment of the invention the diluent is an aqueous diluent. In a further preferred embodiment, the aqueous diluent comprises at least one buffering agent. In a further preferred embodiment, the diluent is an aqueous diluent comprising at least one buffering agent wherein the at least one buffering agent is adapted to maintain a pH from about 6.5 to about 7.5, preferably a pH about 7.0. In a further preferred embodiment, the diluent is an aqueous diluent comprising a phosphate buffer and has a pH from about 11.0 to about 13.5, preferably from about 13.0 to about 13.5, more preferably about 13.1. Usually the aqueous diluent is prepared by mixing a phosphate buffer, preferably a 200 mM phosphate buffer with pH 7 with NaOH 1N and water for injection. The dilution of the liquid composition comprising from about 20 mg/mL to about 30 mg/mL of the active compound cyclo(-Thr-Trp-Ile-Dab-Orn-^{D}Dab-Dab-Trp-Dab-Dab-Ala-Ser-^{D}Pro-Pro-) can be further diluted with e.g. saline (0.90% w/v of NaCl) to arrive at the pharmaceutical composition comprising the active compound cyclo(-Thr-Trp-Ile-Dab-Orn-^{D}Dab-Dab-Trp-Dab-Dab-Ala-Ser-^{D}Pro-Pro-) of the present invention; or any pharmaceutically acceptable salt thereof;
and optionally one or more pharmaceutically acceptable diluents, excipients or carriers; wherein the pharmaceutical composition comprises from about 1.5 mg/mL to about 25 mg/mL of the active compound cyclo(-Thr-Trp-Ile-Dab-Orn-^{D}Dab-Dab-Trp-Dab-Dab-Ala-Ser-D Pro-Pro-).

Thus in a further aspect, the present invention provides a pharmaceutical composition comprising the active compound cyclo(-Thr-Trp-Ile-Dab-Orn-^{D}Dab-Dab-Trp-Dab-Dab-Ala-Ser-^{D}Pro-Pro-);
or any pharmaceutically acceptable salt thereof;
and optionally one or more pharmaceutically acceptable diluents, excipients or carriers; wherein the pharmaceutical composition comprises from about 1.5 mg/mL to about 25 mg/mL of the active compound cyclo(-Thr-Trp-Ile-Dab-Orn-^{D}Dab-Dab-Trp-Dab-Dab-Ala-Ser-^{D}Pro-Pro-).

In a preferred embodiment, the pharmaceutical composition comprises the acetate salt of the active compound cyclo(-Thr-Trp-Ile-Dab-Orn-^{D}Dab-Dab-Trp-Dab-Dab-Ala-Ser-^{D}Pro-Pro-); and optionally one or more pharmaceutically acceptable diluents, excipients or carriers.

In a further preferred embodiment the pharmaceutical composition comprises a dilution of a liquid composition comprising from about 40 mg/mL to about 60 mg/mL of the active compound cyclo(-Thr-Trp-Ile-Dab-Orn-^{D}Dab-Dab-Trp-Dab-Dab-Ala-Ser-^{D}Pro-Pro-), wherein the composition has a pH from about 3.5 to about 4.5, wherein the dilution comprises from about 20 mg/mL to about 30 mg/mL of the active compound cyclo(-Thr-Trp-Ile-Dab-Orn-^{D}Dab-Dab-Trp-Dab-Dab-Ala-Ser-^{D}Pro-Pro-), wherein the dilution has a pH from about 6.5 to about 7.5, preferably a pH of about 7.0, and optionally a osmolality from about 150 to about 500 mOsm/kg, preferably from about 200 to about 400 mOsm/kg, and wherein the dilution is further diluted to obtain a pharmaceutical composition comprising about 1.5 mg/mL to about 25 mg/mL of the active compound cyclo(-Thr-Trp-Ile-Dab-Orn-^{D}Dab-Dab-Trp-Dab-Dab-Ala-Ser-^{D}Pro-Pro-).

In a preferred embodiment the dilution is further diluted by adding an aqueous solution, preferably by adding saline (0.90% w/v of NaCl).

In a further preferred embodiment the pharmaceutical composition comprising about 1.5 mg/mL to about 25 mg/mL of the active compound cyclo(-Thr-Trp-Ile-Dab-Orn-^{D}Dab-Dab-Trp-Dab-Dab-Ala-Ser-^{D}Pro-Pro-) has an osmolality of about 150 to about 500 mOsm/kg, preferably from about 200 to about 400 mOsm/kg.

In a further aspect, the present invention provides a pharmaceutical composition comprising the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof;
and optionally one or more pharmaceutically acceptable diluents, excipients or carriers; wherein the pharmaceutical composition comprises from about 1.5 mg/mL to about 25 mg/mL of the active compound cyclo(-Thr-Trp-Ile-Dab-Orn-^{D}Dab-Dab-Trp-Dab-Dab-Ala-Ser-^{D}Pro-Pro-) for use in a method for the prevention, management or treatment of diseases or conditions of the lungs in a subject.

In one embodiment, the diseases or conditions of the lungs are biofilm-associated diseases or conditions of the lungs.

The present invention also provides a pharmaceutical composition comprising the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof;
and optionally one or more pharmaceutically acceptable diluents, excipients or carriers;

wherein the pharmaceutical composition comprises from about 1.5 mg/mL to about 25 mg/mL of the active compound cyclo(-Thr-Trp-Ile-Dab-Orn-^{D}Dab-Dab-Trp-Dab-Dab-Ala-Ser-^{D}Pro-Pro-), for use in a method for eradicating a biofilm in biofilm-associated diseases or conditions of the lungs in a subject.

The present invention also provides a pharmaceutical composition comprising the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof;

and optionally one or more pharmaceutically acceptable diluents, excipients or carriers;

wherein the pharmaceutical composition comprises from about 1.5 mg/mL to about 25 mg/mL of the active compound cyclo(-Thr-Trp-Ile-Dab-Orn-^{D}Dab-Dab-Trp-Dab-Dab-Ala-Ser-^{D}Pro-Pro-), for use in a method for eradicating a biofilm in biofilm-associated diseases or conditions.

The present invention also provides a pharmaceutical composition comprising the active compound cyclo(-Thr-Trp-Ile-Dab-Orn-^{D}Dab-Dab-Trp-Dab-Dab-Ala-Ser-^{D}Pro-Pro-);
or any pharmaceutically acceptable salt thereof;
and optionally one or more pharmaceutically acceptable diluents, excipients or carriers;
wherein the pharmaceutical composition comprises from about 1.5 mg/mL to about 25 mg/mL of the active compound cyclo(-Thr-Trp-Ile-Dab-Orn-^{D}Dab-Dab-Trp-Dab-Dab-Ala-Ser-^{D}Pro-Pro-), for use in a method for the prevention, management or treatment of biofilm formation.

Also provided is the use of the pharmaceutical composition as described herein for the manufacture of a medicament for the prevention, management or treatment of diseases or conditions of the lungs in a subject, for eradicating a biofilm in biofilm-associated diseases or conditions of the lungs in a subject, for eradicating a biofilm in biofilm-associated diseases or conditions, or for the prevention, management or treatment of biofilm formation, preferably for the manufacture of a medicament for the treatment of diseases or conditions of the lungs in a subject.

Also provided is the use of the pharmaceutical composition as described herein for the prevention, management or treatment of diseases or conditions of the lungs in a subject, for eradicating a biofilm in biofilm-associated diseases or conditions of the lungs in a subject, for eradicating a biofilm in biofilm-associated diseases or conditions, or for the prevention, management or treatment of biofilm formation, preferably for the treatment of diseases or conditions of the lungs in a subject.

Also provided is a method for the prevention, management or treatment of diseases or conditions of the lungs in a subject, for eradicating a biofilm in biofilm-associated diseases or conditions of the lungs in a subject, for eradicating a biofilm in biofilm-associated diseases or conditions, or for the prevention, management or treatment of biofilm formation, preferably a method for the treatment of diseases or conditions of the lungs in a subject, comprising administering to said subject the pharmaceutical composition as described herein e.g. administering to said subject a therapeutically effective amount of a pharmaceutical composition as described herein.

In a preferred embodiment the diseases or conditions of the lungs are selected from the group consisting of cystic fibrosis (CF), non-cystic fibrosis bronchiectasis (NCFB), asthma, chronic obstructive pulmonary disease (COPD), ventilator-associated pneumonia (VAP), ventilator-associated bacterial pneumonia (VABP), hospital-acquired pneumonia (HAP), hospital-acquired bacterial pneumonia (HABP), and healthcare-associated pneumonia (HCAP). In a more preferred embodiment, the diseases or conditions of the lungs is cystic fibrosis (CF), non-cystic fibrosis bronchiectasis (NCFB), asthma, or chronic obstructive pulmonary disease (COPD). In an even more preferred embodiment, the diseases or conditions of the lungs is cystic fibrosis (CF), or non-cystic fibrosis bronchiectasis (NCFB). In a particular preferred embodiment, the diseases or conditions of the lungs is cystic fibrosis (CF).

In a further preferred embodiment, the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof is administered to the subject as pharmaceutical aerosol for pulmonary administration comprising a dispersed liquid phase and a continuous gas phase, wherein the dispersed liquid phase
(a) comprises aqueous droplets comprising the active compound cyclo(-Thr-Trp-Ile-Dab-Orn-^{D}Dab-Dab-Trp-Dab-Dab-Ala-Ser-^{D}Pro-Pro-);
or any pharmaceutically acceptable salt thereof;
(b) has a mass median aerodynamic diameter from about 1.5 μm to about 5 μm; and
(c) has a droplet size distribution having a geometrical standard deviation from about 1.2 to about 1.8.

In a further preferred embodiment, the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof; is administered to the subject at a dose between about 5 and about 500 mg/day, or between about 12 and about 420 mg/day, or between about 100 and about 200 mg/day of the active compound cyclo(-Thr-Trp-Ile-Dab-Orn-^{D}Dab-Dab-Trp-Dab-Dab-Ala-Ser-^{D}Pro-Pro-).

In a more preferred embodiment, the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof; is administered to the subject by oral inhalation.

Thus in a further aspect, the present invention provides a pharmaceutical aerosol for pulmonary administration comprising a dispersed liquid phase and a continuous gas phase, wherein the dispersed liquid phase
(a) comprises aqueous droplets comprising the active compound cyclo(-Thr-Trp-Ile-Dab-Orn-^{D}Dab-Dab-Trp-Dab-Dab-Ala-Ser-^{D}Pro-Pro-);
or any pharmaceutically acceptable salt thereof;
(b) has a mass median aerodynamic diameter from about 1.5 μm to about 5 μm; and
(c) has a droplet size distribution having a geometrical standard deviation from about 1.2 to about 1.8.

In a preferred embodiment, the pharmaceutical aerosol for pulmonary administration comprises from about 1.5 mg/mL to about 25 mg/mL of the active compound cyclo(-Thr-Trp-Ile-Dab-Orn-^{D}Dab-Dab-Trp-Dab-Dab-Ala-Ser-^{D}Pro-Pro-).

In a further preferred embodiment, the dispersed liquid phase of the pharmaceutical aerosol has an osmolality of 150 to about 500 mOsm/kg, preferably from about 200 to about 400 mOsm/kg.

In a further aspect, the present invention provides the pharmaceutical aerosol for pulmonary administration mentioned supra, for use in a method for the prevention, management or treatment of diseases or conditions of the lungs in a subject.

In one embodiment, the diseases or conditions of the lungs are biofilm-associated diseases or conditions of the lungs.

The present invention also provides the pharmaceutical aerosol for pulmonary administration mentioned supra, for use in a method for eradicating a biofilm in biofilm-associated diseases or conditions of the lungs in a subject.

The present invention also provides the pharmaceutical aerosol for pulmonary administration mentioned supra, for use in a method for eradicating a biofilm in biofilm-associated diseases or conditions.

The present invention also the pharmaceutical aerosol for pulmonary administration mentioned supra, for use in a method for the prevention, management or treatment of biofilm formation.

In a preferred embodiment the diseases or conditions of the lungs are selected from the group consisting of cystic fibrosis (CF), non-cystic fibrosis bronchiectasis (NCFB), asthma, chronic obstructive pulmonary disease (COPD), ventilator-associated pneumonia (VAP), ventilator-associated bacterial pneumonia (VABP), hospital-acquired pneumonia (HAP), hospital-acquired bacterial pneumonia (HABP), and healthcare-associated pneumonia (HCAP). In a more preferred embodiment, the diseases or conditions of the lungs is cystic fibrosis (CF), non-cystic fibrosis bronchiectasis (NCFB), asthma, or chronic obstructive pulmonary disease (COPD). In an even more preferred embodiment, the diseases or conditions of the lungs is cystic fibrosis (CF), or non-cystic fibrosis bronchiectasis (NCFB). In a particular preferred embodiment, the diseases or conditions of the lungs is cystic fibrosis (CF).

In a preferred embodiment the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof; is administered to the subject at a dose between about 5 and about 500 mg/day, or between about 12 and about 420 mg/day, or between about 100 and about 200 mg/day of the active compound cyclo(-Thr-Trp-Ile-Dab-Orn-^{D}Dab-Dab-Trp-Dab-Dab-Ala-Ser-^{D}Pro-Pro-).

In a preferred embodiment the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof is administered to the subject at a dose between about 0.03 and about 7.2 mg/kg, or between about 0.08 and about 6 mg/kg, or between about 0.7 and about 2.8 mg/kg of the active compound cyclo(-Thr-Trp-Ile-Dab-Orn-^{D}Dab-Dab-Trp-Dab-Dab-Ala-Ser-^{D}Pro-Pro-).

In a preferred embodiment the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof; is administered to the subject by oral inhalation.

The aerosol of the invention is for pulmonary delivery, which is preferable achieved via oral inhalation of the aerosol. As used herein in the description and the claims, pulmonary delivery means aerosol delivery to any part or feature of the lungs including the so-called deep lungs, the peripheral lungs, the alveoli, the bronchi and the bronchioli.

Conditions of the pulmonary target regions in which the prevention, management or treatment of mammals, more preferably, of human subjects, using the aerosol of the invention is potentially useful include diseases or conditions of the lungs, e.g. in particular, pulmonary diseases, such as cystic fibrosis (CF), non-cystic fibrosis bronchiectasis (NCFB), asthma, chronic obstructive pulmonary disease (COPD), ventilator-associated pneumonia (VAP), ventilator-associated bacterial pneumonia (VABP), hospital-acquired pneumonia (HAP), hospital-acquired bacterial pneumonia (HABP), or healthcare-associated pneumonia (HCAP).

As used herein in the description and the claims, an aerosol is a dispersion of a solid and/or liquid phase in a gas phase. The dispersed phase, also termed discontinuous phase, comprises multiple solid and/or liquid particles. Both basic physical types of aerosols, i.e. solid and liquid dispersions in a gas phase, may be used as pharmaceutical aerosols.

According to the present invention, the aerosol comprises a dispersed liquid phase and a continuous gas phase. Such aerosols are sometimes referred to as “liquid aerosols” or aerosolized liquids. It should be noted that the requirement of a dispersed liquid phase does not exclude the presence of a solid phase. In particular, the dispersed liquid phase may itself represent a dispersion, such as a suspension of solid particles in a liquid.

The continuous gas phase is to be selected from any gas or mixture of gases which is pharmaceutically acceptable. For example, air or compressed air as gas phase is most common in inhalation therapy using nebulizers as aerosol generators. Alternatively, other gases and gas mixtures, such as air enriched with oxygen, or mixtures of nitrogen and oxygen may be used. The use of air as continuous gas phase is most preferred.

The aerosol is further characterized in that the droplets of the dispersed liquid phase have a mass median aerodynamic diameter from about 1.5 μm to about 5 μm with a droplet size distribution having a geometrical standard deviation from about 1.2 to about 1.8. The mass median aerodynamic diameter (MMAD), as used herein in the description and the claims, is the mass median aerodynamic diameter of the dispersed liquid phase as measured by impactor measurement (Next Generation Impactor, NGI) according to US Pharmacopeia (USP) chapter <1601> respectively European Pharmacopoeia (Ph. Eur). 2.9.18. The geometric distribution including the geometric standard deviation (GSD) of the aerosolized liquid particles or droplets may be determined simultaneously with the MMAD. The GSD describes how spread out is a set of numbers the preferred average of which is the geometric mean.

In another preferred embodiment, the aerosol is emitted from an aerosol generator at a mean delivery rate of at least about 0.2 mg of the active compound cyclo(-Thr-Trp-Ile-Dab-Orn-^{D}Dab-Dab-Trp-Dab-Dab-Ala-Ser-^{D}Pro-Pro), or any pharmaceutically acceptable salt thereof, per minute. The (mean) delivery rate of a drug or active compound is one of two discrete metrics or parameters being defined and measured according to e.g. Ph. Eur. (Pharmacopeia Europaea) 2.9.44 and/or USP (United States Pharmacopeia) 1601 to determine the amount of drug or active compound a patient might be expected to receive during a treatment period. In further embodiments, the mean delivery rate of the active compound cyclo(-Thr-Trp-Ile-Dab-Orn-^{D}Dab-Dab-Trp-Dab-Dab-Ala-Ser-^{D}Pro-Pro), or any pharmaceutically acceptable salt thereof, is within the range from about 0.3 mg to about 6 mg per minute.

Appropriate aerosol generators, in particular nebulizers, which are suitable for generating the aerosol(s) described herein in the description and the claims are discussed in more detail herein-below.

Nebulizers are devices capable of aerosolizing liquids. Preferably, the nebulizer of the kit of the invention is selected from jet nebulizers, ultrasonic nebulizers, piezoelectric nebulizers, jet collision nebulizers, electrohydrodynamic nebulizers, capillary force nebulizers, perforated membrane nebulizers and perforated vibrating membrane nebulizers (M. Knoch, M. Keller, Expert Opin. Drug Deliv., 2005, 2, 377). Particularly preferred are piezoelectric, electrohydrodynamic and/or perforated membrane-type nebulizers, e.g. nebulizers from the drug delivery platforms Mystic™ (Battelle Pharma [Battelle Memorial Institute], United States), eFlow™ (Pari GmbH, Starnberg, Germany), Aeroneb™, Aeroneb Pro™, Aero Dose™ (Aerogen Inc, United States) with eFlow™ (Pari GmbH, Starnberg, Germany) most preferred. These types of nebulizers are particularly useful if the aerosol is to be delivered to the bronchi and/or lungs.

The nebulizer should preferably be selected or adapted to be capable of aerosolizing and emitting the liquid composition at a mean delivery rate of at least about 0.2 mg of the active compound cyclo(-Thr-Trp-Ile-Dab-Orn-^{D}Dab-Dab-Trp-Dab-Dab-Ala-Ser-^{D}Pro-Pro), or any pharmaceutically acceptable salt thereof, per minute. The (mean) delivery rate of a drug or active compound is a parameter to determine the amount of drug or active compound a patient might be expected to receive during a treatment period. In further embodiments, the nebulizer is selected or adapted to enable a mean delivery rate of the active compound cyclo(-Thr-Trp-Ile-Dab-Orn-^{D}Dab-Dab-Trp-Dab-Dab-Ala-Ser-^{D}Pro-Pro), or any pharmaceutically acceptable salt thereof, at the range from about 0.3 mg to about 6 mg per minute.

According to a further preference, the nebulizer should be selected or adapted to be capable of aerosolizing and emitting at least of about 50 wt.-% of the loaded dose of the active compound cyclo(-Thr-Trp-Ile-Dab-Orn-^{D}Dab-Dab-Trp-Dab-Dab-Ala-Ser-^{D}Pro-Pro), or any pharmaceutically acceptable salt thereof, whereas said fraction of the loaded dose is comprised of droplets having a mass median aerodynamic diameter of not more than about 5 μm. A fraction of a dispersed phase having a droplet size of not more than about 5 μm is often referred to as the respirable fraction, as droplets of said size—in contrast to larger droplets—have a high chance of being deposited in the lungs, instead of the trachea and the pharynx. More preferably, at least of about 70 wt.-% of the dose filled into the nebulizer is aerosolized to droplets of a size of not more than about 5 μm and emitted from the device. Such a device may be best selected by using an, optionally customized, electronic nebulizer based on the vibrating perforated membrane design, such as a nebulizer from the eFlow™ drug delivery platform (Pari GmbH, Starnberg, Germany).

As defined herein in the description and the claims, a pharmaceutical composition is a liquid material which comprises usually at least one active compound and at least one pharmaceutically acceptable, pharmacologically substantially inert excipient. It should be noted that the term “liquid material” does not necessarily mean that no solid material is present. For example, a liquid suspension representing a dispersion of solid particles in a continuous liquid phase is also embraced in the above term.

Preferably, the pharmaceutical composition from which the aerosol is prepared is an aqueous composition; consequently, water is the predominant liquid constituent of such composition. Solvents and co-solvents other than water should be avoided. In another embodiment, the composition comprises at least about 80 wt.-% of water. In yet another embodiment, at least about 90 wt.-% of the liquid constituents of the composition is water.

If the incorporation of a non-aqueous solvent, such as ethanol, glycerol, propylene glycol or polyethylene glycol, cannot be avoided, the excipient should be selected carefully and in consideration of its physiological acceptability and the therapeutic use of the composition. According to a preferred embodiment, the composition is substantially free of non-aqueous solvents.

The dynamic viscosity of the pharmaceutical composition to prepare the aerosol and the dynamic viscosity of the liquid phase of the aerosol, respectively, has an influence on the efficiency of nebulization and on the particle size distribution of the aerosol formed by nebulization. The dynamic viscosity should preferably be adjusted to a range from about 0.8 mPas*s to about 1.7 mPas*s.

In order to obtain an aerosol which is highly suitable for pulmonary administration, the surface tension of the pharmaceutical composition and the liquid phase of the aerosol, respectively, of the invention should preferably be adjusted to a range from about 25 mN/m to about 80 mN/m.

It is well known in the art that addition of a surfactant to an aqueous liquid composition may result in a surface tension being reduced fairly markedly below that of water or physiological buffer solution. Therefore, a compromise has to be found in each case depending on the intended application.

In order to be well-tolerated an aerosol should—as far as possible—have a physiological tonicity or osmolality. Thus, it may be desirable to incorporate an osmotically active excipient to control the osmolality of the aerosol. Such an excipient, or excipients, if e.g. a combination of substances is used, should be selected to ideally reach an osmolality of the aerosol which does not deviate too much from that of physiological fluids, i.e. from about 150 mOsmol/kg. However, a compromise has again to be found between the physical-chemical and/or pharmaceutical needs on one hand and the physiological requirements on the other hand. In general, an osmolality up to about 800 mOsmol/kg may be acceptable. In particular, an osmolality in the range from about 150 mOsmol/kg to about 500 mOsmol/kg is preferred, a more preferred range is from about 200 mOsmol/kg to about 400 mOsmol/kg, respectively.

The pharmaceutical composition of the invention may comprise further pharmaceutically acceptable excipients, e.g. osmotic agents, such as inorganic salts; excipients for adjusting and/or buffering the pH, such as organic or inorganic salts, acids and bases, bulking agents and lyophilisation aids, such as sucrose and lactose, sugar alcohols, like mannitol, sorbitol, and xylitol, stabilizers and antioxidants, such as vitamin E including its derivatives, lycopene including its derivatives and ascorbic acid, ionic and non-ionic surfactants, such as phospholipids and polysorbates, taste-modifying agents, disintegrants, colouring agents, sweeteners, and/or flavours.

In one of the preferred embodiments, one or more osmotic agents, such as sodium chloride, are incorporated in the pharmaceutical composition to adjust the osmolality to a value in a preferred range as outlined herein above. In a more preferred embodiment, the osmotic agent is sodium chloride.

In order to provide a well tolerated aerosol, the preparation according to the invention should be adjusted to a euhydric pH. The term “euhydric” implies that there may be a difference between pharmaceutical and physiological requirements so that a compromise has to be found which, for example, ensures that, on one hand, the preparation is sufficiently stable during storage, but, on the other hand, is still well tolerated. Preferably, the pH value lies in the slightly acidic to neutral region, i.e. between about 4 and about 8. In general, deviations towards a weakly acidic milieu are tolerated better than an alkaline shift. Particularly preferred is a composition having a pH lying within the range from about 4.5 and about 7.5. preferably about 7.0.

For adjusting the pH of the pharmaceutical composition of the invention and/or buffering such composition, physiologically acceptable acids, bases, salts, and combination of these may be used. Suitable excipients for lowering the pH value and/or as acidic components of a buffer system are strong mineral acids, such as sulphuric acid and hydrochloric acid. Inorganic and organic acids of medium strength, such as phosphoric acid, citric acid, tartaric acid, succinic acid, fumaric acid, methionine, lactic acid, acetic acid, glucuronic acid, as well as acidic salts, such as hydrogen phosphates with sodium or potassium, may be used as well. Suitable excipients for raising the pH value and/or as basic components of a buffer system are mineral bases, such as sodium hydroxide, or other alkali and alkaline earth hydroxides and oxides, such as magnesium hydroxide, calcium hydroxide, or basic ammonium salts, such as ammonium hydroxide, ammonium acetate, or basic amino acids, such as lysine, or carbonates, such as sodium or magnesium carbonate, sodium hydrogen carbonate, or citrates, such as sodium citrate.

In a preferred embodiment, the pharmaceutical composition of the invention comprises at least one excipient to adjust the pH. In a more preferred embodiment, that excipient is sodium hydroxide.

Mainly for pharmaceutical reasons the chemical stabilisation of the pharmaceutical composition of the invention by further additives may be indicated. The most common degradation reactions of a chemically defined active compound in aqueous preparations comprise, in particular, hydrolysis reactions which may be limited primarily by optimal pH adjustment, as well as oxidation reactions. As the active compound cyclo(-Thr-Trp-Ile-Dab-Orn-^{D}Dab-Dab-Trp-Dab-Dab-Ala-Ser-^{D}Pro-Pro) comprises a ornithine residue having a primary amino group, the latter, for example, may be subject to oxidative attack. Therefore, the addition of an antioxidant, or an antioxidant in combination with a synergist, may be advisable or necessary.

Antioxidants are natural or synthetic substances which are capable of preventing or inhibiting the oxidation of the active compound. Antioxidants are primarily adjuvants which are oxidizable and/or act as reducing agents, such as tocopherol acetate, lycopene, reduced glutathione, catalase, peroxide dismutase. Further suitable antioxidants are, for example, ascorbic acid, sodium ascorbate and other salts and derivatives of ascorbic acid, e.g. ascorbyl palmitate, fumaric acid and its salts, malic acid and its salts.

Synergistic substances are those which do not directly act as reactants in oxidation processes, but which counteract such processes by indirect mechanisms, for example, by complexation of metal ions which are known to act catalytically in oxidation processes. Ethylenediaminetetraacetic acid (EDTA) and salts and derivatives thereof, citric acid and salts thereof, malic acid and salts thereof, are such synergistic substances which may act as chelating agents.

In one of the embodiments, the pharmaceutical composition of the invention comprises at least one antioxidant. In a further embodiment, the composition comprises both an antioxidant and a chelating agent.

The pharmaceutical composition of the invention may comprise an excipient affecting the taste. A bad taste is extremely unpleasant and irritating, especially in inhalation administration, and can result in non-compliance, and thus, therapy failure. The bad taste is perceived by the patient through that part of the aerosol which precipitates in the oral and pharyngeal region during inhalation. Even if the particle size of the aerosol can be optimized in such a manner that only a small fraction of the preparation precipitates in the above mentioned regions (said fraction being lost for therapy, unless the oral, pharyngeal or nasal mucosa is the target tissue) it is presently hardly possible to reduce said fraction to such an extent that the bad taste of an active compound is no longer perceived. Therefore, the improvement of the taste of a composition or the masking of the taste of an active compound may be crucial.

In order to improve the taste of the pharmaceutical composition, one or more potentially useful excipients from the group of sugars, sugar alcohols, salts, flavours, complexing agents, polymers, sweeteners, such as sodium saccharin, aspartame, surfactants may be incorporated.

In another aspect the invention provides a method of preparing and delivering an aerosol for pulmonary administration, said method comprising the steps of providing a pharmaceutical composition as described supra comprising the active compound cyclo(-Thr-Trp-Ile-Dab-Orn-^{D}Dab-Dab-Trp-Dab-Dab-Ala-Ser-^{D}Pro-Pro), or any pharmaceutically acceptable salt thereof, in liquid form, wherein the pharmaceutical composition comprises from about 1.5 mg/mL to about 25 mg/mL of the active compound cyclo(-Thr-Trp-Ile-Dab-Orn-^{D}Dab-Dab-Trp-Dab-Dab-Ala-Ser-^{D}Pro-Pro-) and providing a nebulizer capable of aerosolizing said liquid pharmaceutical composition.

The pharmaceutical composition, the pharmaceutical aerosol, or the kit of the invention can be used for the prevention, management or treatment of diseases or conditions of the lungs selected from the group consisting of cystic fibrosis (CF), non-cystic fibrosis bronchiectasis (NCFB), asthma, chronic obstructive pulmonary disease (COPD), ventilator-associated pneumonia (VAP), ventilator-associated bacterial pneumonia (VABP), hospital-acquired pneumonia (HAP), hospital-acquired bacterial pneumonia (HABP), and healthcare-associated pneumonia (HCAP).

As used herein, the term “prevention”/“preventing” e.g. preventive treatments comprise prophylactic treatments. In preventive applications, the pharmaceutical composition or the pharmaceutical aerosol of the invention is administered to a subject suspected of having, or at risk for developing diseases or conditions of the lungs.

As used herein, the term “management” means increasing the time to appearance of a symptom of diseases or conditions of the lungs or a mark associated with diseases or conditions of the lungs or slowing the increase in severity of a symptom of diseases or conditions of the lungs. Further, “management” as used herein includes reversing or inhibition of disease progression or reversing or inhibition of biofilm formation. “Inhibition” of disease progression or disease complication in a subject means preventing or reducing the disease progression and/or disease complication in the subject.

The terms “treatment”/“treating” as used herein includes: (1) delaying the appearance of clinical symptoms of the state, disease or condition developing in an animal, particularly a mammal and especially a human, that may be afflicted with or predisposed to the state, disease or condition but does not yet experience or display clinical or subclinical symptoms of the state, disease or condition; (2) inhibiting the state or condition (e.g. arresting, reducing or delaying the development of the disease, or a relapse thereof in case of maintenance treatment, of at least one clinical or subclinical symptom thereof; and/or (3) relieving the condition (i.e. causing regression of the state, disease or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment. Further “treatment”/“treating” as it relates to the treatment of biofilm formation” herein includes controlling or reversing of biofilm formation.

In therapeutic applications, the pharmaceutical composition is usually administered to a subject such as a patient already suffering from diseases or conditions of the lungs, in an amount sufficient to cure or at least partially arrest the symptoms of the disease or condition. Amounts effective for this use will depend on the severity and course of the disease, previous therapy, the subject's health status and response to the drugs, and the judgment of the treating physician.

In the case wherein the subject's condition does not improve, the pharmaceutical composition or the pharmaceutical aerosol of the invention may be administered chronically, which is, for an extended period of time, including throughout the duration of the subject's life in order to ameliorate or otherwise control or limit the symptoms of the subject's disease or condition.

In the case wherein the subject's status does improve, the pharmaceutical composition or the pharmaceutical aerosol may be administered continuously; alternatively, the dose of drugs being administered may be temporarily reduced or temporarily suspended for a certain length of time (i.e., a “drug holiday”).

Once improvement of the patient's condition has occurred, a maintenance dose of the pharmaceutical composition or the pharmaceutical aerosol of the invention is administered if necessary. Subsequently, the dosage or the frequency of administration, or both, is optionally reduced, as a function of the symptoms, to a level at which the improved disease is retained.

Thus in another aspect the invention provides a pharmaceutical composition comprising the active compound cyclo(-Thr-Trp-Ile-Dab-Orn-^{D}Dab-Dab-Trp-Dab-Dab-Ala-Ser-^{D}Pro-Pro), or any pharmaceutically acceptable salt thereof; and optionally one or more pharmaceutically acceptable diluents, excipients or carriers, for use in a method for the prevention, management or treatment of diseases or conditions of the lungs in a subject, preferably for use in a method for treatment of diseases or conditions of the lungs in a subject.

Also provided is the use of the pharmaceutical composition as described herein for the manufacture of a medicament for the prevention, management or treatment of diseases or conditions of the lungs in a subject, preferably for the manufacture of a medicament for the treatment of diseases or conditions of the lungs in a subject.

Also provided is the use of a pharmaceutical composition as described herein for the prevention, management or treatment of diseases or conditions of the lungs in a subject, preferably for the treatment of diseases or conditions of the lungs in a subject.

Also provided is a method for the prevention, management or treatment of diseases or conditions of the lungs in a subject, preferably a method for the treatment of diseases or conditions of the lungs in a subject, comprising administering to said subject a pharmaceutical composition as described herein e.g. administering to said subject a therapeutically effective amount of a pharmaceutical composition as described herein.

Determination of a therapeutically effective amount is well within the capability of those skilled in the art, especially in light of the detailed disclosure provided herein. In some embodiments, a therapeutically effective amount of the active compound or a pharmaceutically acceptable salt thereof, may (i) reduce the concentration of active elastase in sputum of a subject, ii) may inhibit the activity of human neutrophil elastase activity in sputum of a subject. In various embodiments, the amount is sufficient to ameliorate, palliate, lessen, and/or delay one or more of symptoms of diseases or conditions of the lungs in a subject.

The therapeutically effective amount may vary depending on the subject, and disease or condition being treated, the weight and age of the subject, the severity of the disease or condition, and the manner of administering, which can readily be determined by one ordinary skilled in the art.

In a preferred embodiment the diseases or conditions of the lungs are selected from the group consisting of cystic fibrosis (CF), non-cystic fibrosis bronchiectasis (NCFB), asthma, chronic obstructive pulmonary disease (COPD), ventilator-associated pneumonia (VAP), ventilator-associated bacterial pneumonia (VABP), hospital-acquired pneumonia (HAP), hospital-acquired bacterial pneumonia (HABP), and healthcare-associated pneumonia (HCAP).

In a more preferred embodiment, the diseases or conditions of the lungs is cystic fibrosis (CF), non-cystic fibrosis bronchiectasis (NCFB), asthma, or chronic obstructive pulmonary disease (COPD).

In an even more preferred embodiment, the diseases or conditions of the lungs is cystic fibrosis (CF), or non-cystic fibrosis bronchiectasis (NCFB).

In a particular preferred embodiment, the diseases or conditions of the lungs is cystic fibrosis (CF).

In one embodiment the active compound cyclo(-Thr-Trp-Ile-Dab-Orn-^{D}Dab-Dab-Trp-Dab-Dab-Ala-Ser-^{D}Pro-Pro), or any pharmaceutically acceptable salt thereof, is administered to the subject as pharmaceutical aerosol for pulmonary administration comprising a dispersed liquid phase and a continuous gas phase, wherein the dispersed liquid phase
(a) comprises aqueous droplets comprising the active compound cyclo(-Thr-Trp-Ile-Dab-Orn-^{D}Dab-Dab-Trp-Dab-Dab-Ala-Ser-^{D}Pro-Pro);
or any pharmaceutically acceptable salt thereof;
(b) has a mass median aerodynamic diameter from about 1.5 μm to about 5 μm; and
(c) has a droplet size distribution having a geometrical standard deviation from about 1.2 to about 1.8.

Preferably the aerosol being emitted from an aerosol generator at a rate of at least about 0.1 mL dispersed liquid phase per minute.

Preferably the aerosol being emitted from an aerosol generator at a mean delivery rate of at least about 0.2 mg of the active compound cyclo(-Thr-Trp-Ile-Dab-Orn-^{D}Dab-Dab-Trp-Dab-Dab-Ala-Ser-^{D}Pro-Pro); or any pharmaceutically acceptable salt thereof; per minute.

The aerosol is preferably emitted from an aerosol generator at a rate and at a mean delivery rate as described in the preferred embodiments above.

In another aspect the invention provides a pharmaceutical aerosol for pulmonary administration comprising a dispersed liquid phase and a continuous gas phase, wherein the dispersed liquid phase
(a) comprises aqueous droplets comprising the active compound cyclo(-Thr-Trp-Ile-Dab-Orn-^{D}Dab-Dab-Trp-Dab-Dab-Ala-Ser-^{D}Pro-Pro); or any pharmaceutically acceptable salt thereof; wherein
(b) has a mass median aerodynamic diameter from about 1.5 μm to about 5 μm; and
(c) has a droplet size distribution having a geometrical standard deviation from about 1.2 to about 1.8;
for use in a method for the prevention, management or treatment of diseases or conditions of the lungs in a subject.

In one embodiment, the diseases or conditions of the lungs are biofilm-associated diseases or conditions of the lungs.

The present invention also provides the pharmaceutical aerosol for pulmonary administration mentioned supra, for use in a method for eradicating a biofilm in biofilm-associated diseases or conditions of the lungs in a subject.

The present invention also provides the pharmaceutical aerosol for pulmonary administration mentioned supra, for use in a method for eradicating a biofilm in biofilm-associated diseases or conditions.

The present invention also the pharmaceutical aerosol for pulmonary administration mentioned supra, for use in a method for the prevention, management or treatment of biofilm formation.

The aerosol is preferably emitted from an aerosol generator at a rate and at a mean delivery rate as described in the preferred embodiments above.

Also provided is the use of the pharmaceutical aerosol as described herein for the manufacture of a medicament for the prevention, management or treatment of diseases or conditions of the lungs in a subject, for eradicating a biofilm in biofilm-associated diseases or conditions of the lungs in a subject, for eradicating a biofilm in biofilm-associated diseases or conditions, or for the prevention, management or treatment of biofilm formation, preferably for the manufacture of a medicament for the treatment of diseases or conditions of the lungs in a subject.

Also provided is the use of the pharmaceutical aerosol as described herein for the prevention, management or treatment of diseases or conditions of the lungs in a subject, for eradicating a biofilm in biofilm-associated diseases or conditions of the lungs in a subject, for eradicating a biofilm in biofilm-associated diseases or conditions, or for the prevention, management or treatment of biofilm formation, preferably for the treatment of diseases or conditions of the lungs in a subject.

Also provided is a method for the prevention, management or treatment of diseases or conditions of the lungs in a subject, for eradicating a biofilm in biofilm-associated diseases or conditions of the lungs in a subject, for eradicating a biofilm in biofilm-associated diseases or conditions, or for the prevention, management or treatment of biofilm formation, preferably a method for the treatment of diseases or conditions of the lungs in a subject, comprising administering to said subject the pharmaceutical aerosol as described herein e.g. administering to said subject a therapeutically effective amount of a pharmaceutical aerosol as described herein.

In a preferred embodiment, the diseases or conditions of the lungs are selected from the group consisting of cystic fibrosis (CF), non-cystic fibrosis bronchiectasis (NCFB), asthma, chronic obstructive pulmonary disease (COPD), ventilator-associated pneumonia (VAP), ventilator-associated bacterial pneumonia (VABP), hospital-acquired pneumonia (HAP), hospital-acquired bacterial pneumonia (HABP), and healthcare-associated pneumonia (HCAP).

In a more preferred embodiment, the diseases or conditions of the lungs is cystic fibrosis (CF), non-cystic fibrosis bronchiectasis (NCFB), asthma, or chronic obstructive pulmonary disease (COPD).

In an even more preferred embodiment, the diseases or conditions of the lungs is cystic fibrosis (CF), or non-cystic fibrosis bronchiectasis (NCFB).

In a particular preferred embodiment, the diseases or conditions of the lungs is cystic fibrosis (CF).

The counter ion of the active compound of the pharmaceutical composition or the pharmaceutical aerosol for use in a method for the prevention, management or treatment of diseases or conditions of the lungs in a subject is as described for the active compound above and is preferably acetate.

The pharmaceutical composition or the pharmaceutical aerosol for use in a method for the prevention, management or treatment of diseases or conditions of the lungs in a subject is usually administered to the subject by oral inhalation or intratracheal, preferably by oral inhalation.

The dosing regimen of the active compound cyclo(-Thr-Trp-Ile-Dab-Orn-^{D}Dab-Dab-Trp-Dab-Dab-Ala-Ser-^{D}Pro-Pro), or any pharmaceutically acceptable salt thereof, comprised by the pharmaceutical composition or the pharmaceutical aerosol, in the methods provided herein may vary depending upon the indication, route of administration, and severity of the condition, for example. Depending on the route of administration, a suitable dose can be calculated according to body weight, body surface area, or organ size. Additional factors that can be taken into account include time and frequency of administration, drug combinations, reaction sensitivities, and tolerance/response to therapy. The amount, e.g. the therapeutically effective amount of the active compound cyclo(-Thr-Trp-Ile-Dab-Orn-^{D}Dab-Dab-Trp-Dab-Dab-Ala-Ser-^{D}Pro-Pro), or a pharmaceutically acceptable salt thereof, may be provided in a single dose or multiple doses to achieve the desired treatment endpoint.

The frequency of dosing will depend on the pharmacokinetic parameters of the active compound cyclo(-Thr-Trp-Ile-Dab-Orn-^{D}Dab-Dab-Trp-Dab-Dab-Ala-Ser-^{D}Pro-Pro), or any pharmaceutically acceptable salt thereof, administered, the route of administration, and the particular disease treated. The dose and frequency of dosing may also depend on pharmacokinetic and pharmacodynamic, as well as toxicity and therapeutic efficiency data. For example, pharmacokinetic and pharmacodynamic information about the active compound or a pharmaceutically acceptable salt thereof, can be collected through preclinical in vitro and in vivo studies, later confirmed in humans during the course of clinical trials. Thus, for the active compound or a pharmaceutically acceptable salt thereof, used in the methods provided herein, a therapeutically effective dose can be estimated initially from biochemical and/or cell-based assays. Then, dosage can be formulated in animal models to achieve a desirable circulating concentration range. As human studies are conducted further information will emerge regarding the appropriate dosage levels and duration of treatment for various diseases and conditions.

Toxicity and therapeutic efficacy of the active compound cyclo(-Thr-Trp-Ile-Dab-Orn-^{D}Dab-Dab-Trp-Dab-Dab-Ala-Ser-^{D}Pro-Pro), or any pharmaceutically acceptable salt thereof, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the “therapeutic index”, which typically is expressed as the ratio LD₅₀/ED₅₀. Compounds that exhibit large therapeutic indices, i.e., the toxic dose is substantially higher than the effective dose, are preferred. The data obtained from such cell culture assays and additional animal studies can be used in formulating a range of dosage for human use. The doses of such compounds lies preferably within a range of circulating concentrations that include the ED₅₀ with little or no toxicity.

An exemplary treatment regime entails administration once daily, twice daily, three times daily, every day, every second day, every third day, every fourth day, every fifth day, every sixth day, twice per week, once per week. The active compound cyclo(-Thr-Trp-Ile-Dab-Orn-^{D}Dab-Dab-Trp-Dab-Dab-Ala-Ser-^{D}Pro-Pro), or any pharmaceutically acceptable salt thereof, is usually administered on multiple occasions. Intervals between single dosages can be, for example, less than a day, a day, two days, three days, four days, five days, six days or a week. The combination of the invention may be given as a continuous uninterrupted treatment. The combination of the invention may also be given in a regime in which the subject receives cycles of treatment (administration cycles) interrupted by a drug holiday or period of non-treatment.

In one embodiment the active compound cyclo(-Thr-Trp-Ile-Dab-Orn-^{D}Dab-Dab-Trp-Dab-Dab-Ala-Ser-^{D}Pro-Pro); or any pharmaceutically acceptable salt thereof; of the pharmaceutical composition or the pharmaceutical aerosol for use in a method for the prevention, management or treatment of diseases or conditions of the lungs in a subject is administered to the subject at a dose between about 0.1 and about 10000 mg/day of the active compound cyclo(-Thr-Trp-Ile-Dab-Orn-^{D}Dab-Dab-Trp-Dab-Dab-Ala-Ser-^{D}Pro-Pro-).

In one embodiment the active compound cyclo(-Thr-Trp-Ile-Dab-Orn-^{D}Dab-Dab-Trp-Dab-Dab-Ala-Ser-^{D}Pro-Pro); or any pharmaceutically acceptable salt thereof; of the pharmaceutical composition or the pharmaceutical aerosol for use in a method for the prevention, management or treatment of diseases or conditions of the lungs in a subject is administered to the subject at a dose between about 1 and about 1000 mg/day of the active compound cyclo(-Thr-Trp-Ile-Dab-Orn-^{D}Dab-Dab-Trp-Dab-Dab-Ala-Ser-^{D}Pro-Pro-).

In one embodiment the active compound cyclo(-Thr-Trp-Ile-Dab-Orn-^{D}Dab-Dab-Trp-Dab-Dab-Ala-Ser-^{D}Pro-Pro); or any pharmaceutically acceptable salt thereof; of the pharmaceutical composition or the pharmaceutical aerosol for use in a method for the prevention, management or treatment of diseases or conditions of the lungs in a subject is administered to the subject at a dose between about 5 and about 500 mg/day of the active compound cyclo(-Thr-Trp-Ile-Dab-Orn-^{D}Dab-Dab-Trp-Dab-Dab-Ala-Ser-^{D}Pro-Pro-).

In one embodiment the active compound cyclo(-Thr-Trp-Ile-Dab-Orn-^{D}Dab-Dab-Trp-Dab-Dab-Ala-Ser-^{D}Pro-Pro); or any pharmaceutically acceptable salt thereof; of the pharmaceutical composition or the pharmaceutical aerosol for use in a method for the prevention, management or treatment of diseases or conditions of the lungs in a subject is administered to the subject at a dose between about 12 and about 420 mg/day of the active compound cyclo(-Thr-Trp-Ile-Dab-Orn-^{D}Dab-Dab-Trp-Dab-Dab-Ala-Ser-^{D}Pro-Pro-).

In one embodiment the active compound cyclo(-Thr-Trp-Ile-Dab-Orn-^{D}Dab-Dab-Trp-Dab-Dab-Ala-Ser-^{D}Pro-Pro); or any pharmaceutically acceptable salt thereof; of the pharmaceutical composition or the pharmaceutical aerosol for use in a method for the prevention, management or treatment of diseases or conditions of the lungs in a subject is administered to the subject at a dose between about 100 and about 200 mg/day of the active compound cyclo(-Thr-Trp-Ile-Dab-Orn-^{D}Dab-Dab-Trp-Dab-Dab-Ala-Ser-^{D}Pro-Pro-).

In one embodiment the active compound cyclo(-Thr-Trp-Ile-Dab-Orn-^{D}Dab-Dab-Trp-Dab-Dab-Ala-Ser-^{D}Pro-Pro); or any pharmaceutically acceptable salt thereof; of the pharmaceutical composition or the pharmaceutical aerosol for use in a method for the prevention, management or treatment of diseases or conditions of the lungs in a subject is administered to the subject at a dose of about 12, about 12.5, about 25, about 50, about 75, about 150, about 200 mg, about 400, or about 420 mg/day of the active compound cyclo(-Thr-Trp-Ile-Dab-Orn-^{D}Dab-Dab-Trp-Dab-Dab-Ala-Ser-^{D}Pro-Pro-).

In one embodiment the active compound cyclo(-Thr-Trp-Ile-Dab-Orn-^{D}Dab-Dab-Trp-Dab-Dab-Ala-Ser-^{D}Pro-Pro); or any pharmaceutically acceptable salt thereof; of the pharmaceutical composition or the pharmaceutical aerosol for use in a method for the prevention, management or treatment of diseases or conditions of the lungs in a subject is administered to the subject at a dose of about 100, about 150, or about 200 mg/day of the active compound cyclo(-Thr-Trp-Ile-Dab-Orn-^{D}Dab-Dab-Trp-Dab-Dab-Ala-Ser-^{D}Pro-Pro-).

In another aspect the invention provides a kit
for the preparation and delivery of a pharmaceutical aerosol for pulmonary administration comprising a dispersed liquid phase and a continuous gas phase, wherein the dispersed liquid phase
(a) comprises aqueous droplets comprising the active compound cyclo(-Thr-Trp-Ile-Dab-Orn-^{D}Dab-Dab-Trp-Dab-Dab-Ala-Ser-^{D}Pro-Pro-);
or any pharmaceutically acceptable salt thereof;
(b) has a mass median aerodynamic diameter from about 1.5 μm to about 5 μm; and
(c) has a droplet size distribution having a geometrical standard deviation from about 1.2 to about 1.8;
and wherein the kit comprises a nebulizer and a pharmaceutical composition comprising a concentration within a range from about 1.5 mg/mL to about 25 mg/mL of the active compound.

Usually the liquid composition comprising a concentration within a range from about 1.5 mg/mL to about 25 mg/mL of the active compound is the pharmaceutical composition described supra comprising a concentration within a range from about 1.5 mg/mL to about 25 mg/mL of the active compound. In a preferred embodiment the liquid composition of the aerosol has an osmolality of about 150 to about 500 mOsm/kg, preferably of about 200 to about 400 mOsm/kg. In a further preferred embodiment the nebulizer is selected from the group consisting of jet nebulizers, ultrasonic nebulizers, piezoelectric nebulizers, jet collision nebulizers, electrohydrodynamic nebulizers, capillary force nebulizers, perforated membrane nebulizers and perforated vibrating membrane nebulizers as described supra.

In a further preferred embodiment the kit further comprises a package insert wherein the package insert comprises instructions for treating a subject for diseases or conditions of the lungs.

In another aspect the invention provides a kit
for the preparation and delivery of a pharmaceutical aerosol for pulmonary administration comprising a dispersed liquid phase and a continuous gas phase, wherein the dispersed liquid phase
(a) comprises aqueous droplets comprising the active compound cyclo(-Thr-Trp-Ile-Dab-Orn-^{D}Dab-Dab-Trp-Dab-Dab-Ala-Ser-^{D}Pro-Pro-);
or any pharmaceutically acceptable salt thereof;
(b) has a mass median aerodynamic diameter from about 1.5 μm to about 5 μm; and
(c) has a droplet size distribution having a geometrical standard deviation from about 1.2 to about 1.8;
and wherein the kit comprises a nebulizer and a pharmaceutical composition comprising a concentration within a range from about 1.5 mg/mL to about 25 mg/mL of the active compound
for use in a method for the prevention, management or treatment of diseases or conditions of the lungs in a subject.

In one embodiment, the diseases or conditions of the lungs are biofilm-associated diseases or conditions of the lungs.

The present invention also provides a kit
for the preparation and delivery of a pharmaceutical aerosol for pulmonary administration comprising a dispersed liquid phase and a continuous gas phase, wherein the dispersed liquid phase
(a) comprises aqueous droplets comprising the active compound cyclo(-Thr-Trp-Ile-Dab-Orn-^{D}Dab-Dab-Trp-Dab-Dab-Ala-Ser-^{D}Pro-Pro-);
or any pharmaceutically acceptable salt thereof;
(b) has a mass median aerodynamic diameter from about 1.5 μm to about 5 μm; and
(c) has a droplet size distribution having a geometrical standard deviation from about 1.2 to about 1.8;
and wherein the kit comprises a nebulizer and a pharmaceutical composition comprising a concentration within a range from about 1.5 mg/mL to about 25 mg/mL of the active compound, for use in a method for eradicating a biofilm in biofilm-associated diseases or conditions of the lungs in a subject.

The present invention also provides a kit
for the preparation and delivery of a pharmaceutical aerosol for pulmonary administration comprising a dispersed liquid phase and a continuous gas phase, wherein the dispersed liquid phase
(a) comprises aqueous droplets comprising the active compound cyclo(-Thr-Trp-Ile-Dab-Orn-^{D}Dab-Dab-Trp-Dab-Dab-Ala-Ser-^{D}Pro-Pro-);
or any pharmaceutically acceptable salt thereof;
(b) has a mass median aerodynamic diameter from about 1.5 μm to about 5 μm; and
(c) has a droplet size distribution having a geometrical standard deviation from about 1.2 to about 1.8;
and wherein the kit comprises a nebulizer and a pharmaceutical composition comprising a concentration within a range from about 1.5 mg/mL to about 25 mg/mL of the active compound, for use in a method for eradicating a biofilm in biofilm-associated diseases or conditions.

The present invention also provides a kit
for the preparation and delivery of a pharmaceutical aerosol for pulmonary administration comprising a dispersed liquid phase and a continuous gas phase, wherein the dispersed liquid phase
(a) comprises aqueous droplets comprising the active compound cyclo(-Thr-Trp-Ile-Dab-Orn-^{D}Dab-Dab-Trp-Dab-Dab-Ala-Ser-^{D}Pro-Pro-);
or any pharmaceutically acceptable salt thereof;
(b) has a mass median aerodynamic diameter from about 1.5 μm to about 5 μm; and
(c) has a droplet size distribution having a geometrical standard deviation from about 1.2 to about 1.8;
and wherein the kit comprises a nebulizer and a pharmaceutical composition comprising a concentration within a range from about 1.5 mg/mL to about 25 mg/mL of the active compound, for use in a method for the prevention, management or treatment of biofilm formation.

In a preferred embodiment the diseases or conditions of the lungs are selected from the group consisting of cystic fibrosis (CF), non-cystic fibrosis bronchiectasis (NCFB), asthma, chronic obstructive pulmonary disease (COPD), ventilator-associated pneumonia (VAP), ventilator-associated bacterial pneumonia (VABP), hospital-acquired pneumonia (HAP), hospital-acquired bacterial pneumonia (HABP), and healthcare-associated pneumonia (HCAP). In a more preferred embodiment, the diseases or conditions of the lungs is cystic fibrosis (CF), non-cystic fibrosis bronchiectasis (NCFB), asthma, or chronic obstructive pulmonary disease (COPD). In an even more preferred embodiment, the diseases or conditions of the lungs is cystic fibrosis (CF), or non-cystic fibrosis bronchiectasis (NCFB). In a particular preferred embodiment, the diseases or conditions of the lungs is cystic fibrosis (CF).

Also provided is the use of the kit as described herein for the manufacture of a medicament for the prevention, management or treatment of diseases or conditions of the lungs in a subject, for eradicating a biofilm in biofilm-associated diseases or conditions of the lungs in a subject, for eradicating a biofilm in biofilm-associated diseases or conditions, or for the prevention, management or treatment of biofilm formation, preferably for the manufacture of a medicament for the treatment of diseases or conditions of the lungs in a subject.

Also provided is the use of the kit as described herein for the prevention, management or treatment of diseases or conditions of the lungs in a subject, for eradicating a biofilm in biofilm-associated diseases or conditions of the lungs in a subject, for eradicating a biofilm in biofilm-associated diseases or conditions, or for the prevention, management or treatment of biofilm formation, preferably for the treatment of diseases or conditions of the lungs in a subject.

Also provided is a method for the prevention, management or treatment of diseases or conditions of the lungs in a subject, for eradicating a biofilm in biofilm-associated diseases or conditions of the lungs in a subject, for eradicating a biofilm in biofilm-associated diseases or conditions, or for the prevention, management or treatment of biofilm formation, preferably a method for the treatment of diseases or conditions of the lungs in a subject, comprising administering to said subject the pharmaceutical aerosol of the kit as described herein e.g. administering to said subject a therapeutically effective amount of a pharmaceutical aerosol of the kit as described herein.

In one embodiment the nebulizer of the kit for use in a method for the prevention, management or treatment of diseases or conditions of the lungs in a subject is selected from the group consisting of jet nebulizers, ultrasonic nebulizers, piezoelectric nebulizers, jet collision nebulizers, electrohydrodynamic nebulizers, capillary force nebulizers, perforated membrane nebulizers and perforated vibrating membrane nebulizers as described supra.

In one embodiment the counter ion of the active compound comprised by the kit is as described for the active compound above and is preferably acetate.

In a further aspect the present invention provides a pharmaceutical composition comprising the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof;
and optionally one or more pharmaceutically acceptable diluents, excipients or carriers; for use in a method for the prevention, management or treatment of biofilm-associated diseases or conditions of the lungs in a subject.

In a preferred embodiment, the pharmaceutical composition comprises the acetate salt of the active compound cyclo(-Thr-Trp-Ile-Dab-Orn-^{D}Dab-Dab-Trp-Dab-Dab-Ala-Ser-^{D}Pro-Pro-); and optionally one or more pharmaceutically acceptable diluents, excipients or carriers.

In a further aspect the present invention provides a pharmaceutical composition comprising the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof;
and optionally one or more pharmaceutically acceptable diluents, excipients or carriers; for use in a method for eradicating a biofilm in biofilm-associated diseases or conditions of the lungs in a subject.

In a preferred embodiment, the pharmaceutical composition comprises the acetate salt of the active compound cyclo(-Thr-Trp-Ile-Dab-Orn-^{D}Dab-Dab-Trp-Dab-Dab-Ala-Ser-^{D}Pro-Pro-); and optionally one or more pharmaceutically acceptable diluents, excipients or carriers.

In one embodiment the diseases or conditions of the lungs or the biofilm-associated disease or condition of the lungs are selected from the group consisting of cystic fibrosis (CF), non-cystic fibrosis bronchiectasis (NCFB), asthma, chronic obstructive pulmonary disease (COPD), ventilator-associated pneumonia (VAP), ventilator-associated bacterial pneumonia (VABP), hospital-acquired pneumonia (HAP), hospital-acquired bacterial pneumonia (HABP), and healthcare-associated pneumonia (HCAP).

In a preferred embodiment, the pharmaceutical composition comprises the acetate salt of the active compound cyclo(-Thr-Trp-Ile-Dab-Orn-^{D}Dab-Dab-Trp-Dab-Dab-Ala-Ser-^{D}Pro-Pro-); and optionally one or more pharmaceutically acceptable diluents, excipients or carriers.

In one embodiment the diseases or conditions of the lungs or the biofilm-associated disease or condition of the lungs are cystic fibrosis (CF), or non-cystic fibrosis bronchiectasis (NCFB).

In one embodiment the diseases or conditions of the lungs or the biofilm-associated disease or condition of the lungs is cystic fibrosis (CF).

In a further aspect the present invention provides a pharmaceutical composition comprising the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof;
and optionally one or more pharmaceutically acceptable diluents, excipients or carriers; for use in a method for eradicating a biofilm in biofilm-associated diseases or conditions in a subject.

In a preferred embodiment, the pharmaceutical composition comprises the acetate salt of the active compound cyclo(-Thr-Trp-Ile-Dab-Orn-^{D}Dab-Dab-Trp-Dab-Dab-Ala-Ser-^{D}Pro-Pro-);
and optionally one or more pharmaceutically acceptable diluents, excipients or carriers.

In a further aspect the present invention provides a pharmaceutical composition comprising the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof;
and optionally one or more pharmaceutically acceptable diluents, excipients or carriers; for use in a method for the prevention, management or treatment of biofilm formation in a subject.

In a preferred embodiment, the pharmaceutical composition comprises the acetate salt of the active compound cyclo(-Thr-Trp-Ile-Dab-Orn-^{D}Dab-Dab-Trp-Dab-Dab-Ala-Ser-^{D}Pro-Pro-); and optionally one or more pharmaceutically acceptable diluents, excipients or carriers.

Also provided is the use of the pharmaceutical composition as described above for the manufacture of a medicament for the prevention, management or treatment of diseases or conditions of the lungs in a subject, for eradicating a biofilm in biofilm-associated diseases or conditions of the lungs in a subject, for eradicating a biofilm in biofilm-associated diseases or conditions, or for the prevention, management or treatment of biofilm formation, preferably for the manufacture of a medicament for the treatment of diseases or conditions of the lungs in a subject.

Also provided is the use of the pharmaceutical composition as described above for the prevention, management or treatment of diseases or conditions of the lungs in a subject, for eradicating a biofilm in biofilm-associated diseases or conditions of the lungs in a subject, for eradicating a biofilm in biofilm-associated diseases or conditions, or for the prevention, management or treatment of biofilm formation, preferably for the treatment of diseases or conditions of the lungs in a subject.

Also provided is a method for the prevention, management or treatment of diseases or conditions of the lungs in a subject, for eradicating a biofilm in biofilm-associated diseases or conditions of the lungs in a subject, for eradicating a biofilm in biofilm-associated diseases or conditions, or for the prevention, management or treatment of biofilm formation, preferably a method for the treatment of diseases or conditions of the lungs in a subject, comprising administering to said subject the pharmaceutical composition as described herein e.g. administering to said subject a therapeutically effective amount of a pharmaceutical composition as described above.

The following Examples illustrate the present invention but are not to be construed as limiting its scope in any way.

### EXAMPLES

### Analytical Methods

### Method A: LC-MS Purity Determination

Instruments used:
HPLC: Thermo Scientific Ultimate 3000RS (Thermo Fisher Scientific Inc., Waltham, Massachusetts, USA)—Binary pump with mixing Chamber 750 μL—DAD detector UV flow cell semi-micro (volume 2.5 μL—flow path 7 mm)—Column oven
Mass spectrometer: Thermo Scientific MSQ Plus
Stationary phase: Waters Acquity UPLC Peptide BEH C18, 130 Å, 1.7 μm, 2.1×150 mm
Mobile phases:
Eluent A: 0.2% (v/v) TFA and 1% (v/v) ACN in water

Preparation: Place approximately 900 mL of water into a 1 L volumetric flask, add 10.0 mL of ACN and 2.0 mL of TFA. Add water to adjust the volume and mix well.
Eluent B: 0.2% (v/v) TFA in ACN

Place approximately 450 mL of ACN into a 500 mL volumetric flask and 1.0 mL of TFA. Add water to adjust the volume and mix well.

### Sample Preparation:

Diluent: corresponding to the initial composition of the gradient: 8% Eluant B/92% Eluant A. The preparation was performed by mixing 92 mL of Eluant A with 8 mL of Eluant B.

Dilution of the initial solution to analyze was carried out to obtain a final net peptide concentration of 0.5 mg/mL.

HPLC method:
Autosampler temperature: 11° C.
Injection volume: 3 μL
Flow: 0.3 mL/min
Column oven temperature: 70° C.

Gradient:

| Time (min) | Eluent B (%) |
|---|---|
| 0.0 | 8.0 |
| 0.5 | 8.0 |
| 18.5 | 31.5 |
| 19.0 | 31.5 |
| 19.1 | 99.0 |
| 21.0 | 99.0 |
| 21.1 | 8.0 |
| 27.9 | 8.0 |

Detection:
UV Wavelength: 220 nm (bandwidth: 10 nm; data collection rate: 5 Hz; response time: 2 s) MS: ESI positive 60 V, 411° C., mass range: [95-2000]

### Method B: pH Measurement

Instrument: Seven Easy from Metier Toledo (Greifensee, Switzerland)
Electrode: Inlab Expert Pro ISM from Metier Toledo
Prior measurements, the pH meter was calibrated using pH 4, pH 7 and pH 10 standard solutions from Metier Toledo.

### Method C: Osmolality Measurements

Instrument: freezing point osmometer Osmomat 3000 from Gonotec (Berlin, Germany) Prior measurements, the osmometer was calibrated using 15 μL of milliQ water (0 mOsm/kg) and standard solutions at respectively 300 and 500 mOsm/kg from Gonotec.

15 μL of each sample to analyze were used for the measurement.

### Method D: Determination of Surface Tension

The measurements of the dynamic surface tension [mN/m] were based on the bubble pressure method.

Instrument: bubble pressure tensiometer SITA online t60 from SITA (Dresden, Germany). Further devices used were a temperature equilibrating vessel, a refrigerated circulator bath, a stand with clamp and a thermometer. Deionised water was used for cleaning and calibration; the measurements were carried out at a temperature of 20.0° C.±1.0° C.

### Method E: Determination of Dynamic Viscosity

The dynamic viscosity (mPa*s) was measured using a rotational rheometer (HAAKE™ RheoStress™ 1 rheometer, Thermo Fisher Scientific, Waltham, MA, USA) with cone-plate-geometry sensor system. Assessment of the dynamic viscosity was done according to USP <1911>, <912> and Ph.Eur. 2.2.10.

### Method F: Breath Simulation

In the breath simulation experiments, the nebulizer was connected to a sinus pump (Pari breath simulator using an eFlow™ nebuliser, header type class 30, Pari GmbH, Starnberg, Germany). The drug containing aerosol droplets were collected on an inspiratory filter. This filter was installed between the nebulizer including the mouthpiece at the front end of the pump. The nebulizer was filled with the formulation and the nebulization is initiated. One filter change was performed after 1 min to determine drug delivery rate. The nebulization was performed until automatic shut-off of the nebulizer. The drug was recovered from the filters with solvent, and the assay analyzed using the HPLC method A.

### Method G: Determination of Aerodynamic Particle Size Distribution (APSD)

Aerodynamic Particle Size Distribution (APSD) was determined using Next Generation Impactor (NGI) according to USP <1601> and Ph. Eur. 2.9.18. The formulation was dispensed in the Pari eFlow™ nebuliser, header type class 30 (Pari GmbH, Starnberg, Germany), and attached to a Next Generation Impactor (NGI) with appropriate pump and flow controller. The nebuliser was actuated at a constant flow through the NGI until empty. The drug was recovered from each stage or component of the NGI with solvent and analyzed using method A.

Determination of the Optimal pH for the Acetate Salt of Cyclo(-Thr-Trp-Ile-Dab-Orn-^{D}Dab-Dab-Trp-Dab-Dab-Ala-Ser-^{D}Pro-Pro-) at 50 mg/mL Net Peptide Content

In order to evaluate which pH leads to the best stability over time, three solutions A examples 1, 2 and 3) were prepared in a laboratory scale of 50 mL at 50 mg/mL net peptide content of the acetate salt of cyclo(-Thr-Trp-Ile-Dab-Orn-^{D}Dab-Dab-Trp-Dab-Dab-Ala-Ser-^{D}Pro-Pro-) (murepavadin) at pH 4, pH 4.5 and pH 4.9, respectively. The purity of each solution at 5° C. and 25° C. was evaluated using LC-MS (method A) over 6 months.

Calculation of the Net Peptide Content of the Acetate Salt of Cyclo(-Thr-Trp-Ile-Dab-Orn-^{D}Dab-Dab-Trp-Dab-Dab-Ala-Ser-^{D}Pro-Pro-) (Drug Substance Comprising Murepavadin as Active Compound):

Net peptide content [%]=[(100−impurity [%]/100)×(100−water content [%]/100)×(100−residual solvent [%]/100)×(100−residual TFA/100)×free salt [%]/100]×100=72.4%.

### Example 1: Preparation of a Solution of the Acetate Salt of Cyclo(-Thr-Trp-Ile-Dab-Orn-^{D}Dab-Dab-Trp-Dab-Dab-Ala-Ser-^{D}Pro-Pro-) at 50 mg/mL Net Peptide Content at pH 4

3.453 g of the acetate salt of cyclo(-Thr-Trp-Ile-Dab-Orn-^{D}Dab-Dab-Trp-Dab-Dab-Ala-Ser-^{D}Pro-Pro-) (drug substance) were dissolved in 30 mL of water for injection (WFI). 6.3 mL of HCl 1N were added to reach pH 4. 13.7 mL of WFI were then added for reaching 50 mL total volume of the solution.
Final pH=4.0 (method B)
Osmolality=355 mOsm/kg (method C)

### Example 2: Preparation of a Solution of the Acetate Salt of Cyclo(-Thr-Trp-Ile-Dab-Orn-^{D}Dab-Dab-Trp-Dab-Dab-Ala-Ser-^{D}Pro-Pro-) at 50 mg/mL Net Peptide Content at pH 4.5

3.453 g of the acetate salt of cyclo(-Thr-Trp-Ile-Dab-Orn-^{D}Dab-Dab-Trp-Dab-Dab-Ala-Ser-D Pro-Pro-) (drug substance) were dissolved in 30 mL of water for injection (WFI). 2.8 mL of HCl 1N were added to reach pH 4.5. 17.2 mL of WFI were then added for reaching 50 mL total volume of the solution.
Final pH=4.5 (method B)
Osmolality=299 mOsm/kg (method C)

### Example 3: Preparation of a Solution of the Acetate Salt of Cyclo(-Thr-Trp-Ile-Dab-Orn-^{D}Dab-Dab-Trp-Dab-Dab-Ala-Ser-^{D}Pro-Pro-) at 50 mg/mL Net Peptide Content at pH 4.9

3.453 g of the acetate salt of cyclo(-Thr-Trp-Ile-Dab-Orn-^{D}Dab-Dab-Trp-Dab-Dab-Ala-Ser-^{D}Pro-Pro-) (drug substance) were dissolved in 50 mL of water for injection (WFI).
Final pH=4.9 (method B)
Osmolality=246 mOsm/kg (method C)

### Stability of the Solutions Described in Examples 1-3 at Different Temperatures Over Time

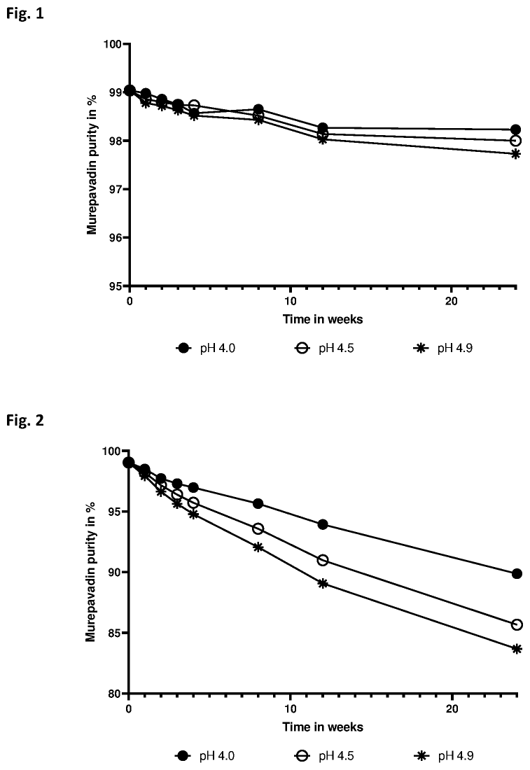

Each solution was partitioned in glass vials and kept at 5° C. or 25° C. These vials were taken at different time points and purity was analyzed using HPLC method A (see Tables 1 and 2 and FIGS. 1 and 2). pH and osmolality were also measured using respectively methods B and C and were stable at each temperature over time. Example 1 (pH 4) showed the best stability at both temperatures (see Table 1 and FIG. 1) and was chosen for determining the neutralization solution.

TABLE 1
Purity of cyclo(-Thr-Trp-Ile-Dab-Orn-^{D}Dab-Dab-Trp-Dab-Dab-Ala-Ser-
^{D}Pro-Pro-), pH and osmolality of the solution over time at 5° C.

| | cyclo(-Thr-Trp-Ile-Dab- | | | | | | | | |
|---|---|---|---|---|---|---|---|---|---|
| | Orn-^{D}Dab-Dab-Trp-Dab- | | | | | | | | |
| | Dab-Ala-Ser-^{D}Pro-Pro-) | | | | | | Osmolality (mOsm/kg) | | |
| Time in | purity (%) | | | pH of the solution | | | of the solution | | |
| weeks | pH 4.9 | pH 4.5 | pH 4.0 | pH 4.9 | pH 4.5 | pH 4.0 | pH 4.9 | pH 4.5 | pH 4.0 |
| 0 | 99.04 | 99.04 | 99.04 | 4.94 | 4.52 | 4.01 | 246 | 299 | 355 |
| 1 | 98.78 | 98.86 | 98.98 | 4.99 | 4.56 | 4.02 | 244 | 294 | 360 |
| 2 | 98.72 | 98.80 | 98.86 | 5.00 | 4.56 | 4.04 | 248 | 297 | 365 |
| 3 | 98.63 | 98.74 | 98.75 | 4.91 | 4.44 | 3.92 | 215 | 266 | 331 |
| 4 | 98.52 | 98.73 | 98.57 | 4.85 | 4.40 | 3.87 | 240 | 292 | 355 |
| 8 | 98.43 | 98.52 | 98.65 | 4.98 | 4.53 | 4.00 | 243 | 291 | 359 |
| 12 | 98.03 | 98.14 | 98.27 | 4.98 | 4.50 | 3.96 | 240 | 291 | 357 |
| 24 | 97.73 | 98.00 | 98.23 | 4.98 | 4.50 | 3.96 | 249 | 312 | 368 |

TABLE 2
Purity of cyclo(-Thr-Trp-Ile-Dab-Orn-^{D}Dab-Dab-Trp-Dab-Dab-Ala-Ser-
^{D}Pro-Pro-), pH and osmolality of the solution over time at 25° C.

| | cyclo(-Thr-Trp-Ile-Dab- | | | | | | | | |
|---|---|---|---|---|---|---|---|---|---|
| | Orn-^{D}Dab-Dab-Trp-Dab- | | | | | | | | |
| | Dab-Ala-Ser-^{D}Pro-Pro-) | | | | | | Osmolality (mOsm/kg) | | |
| Time in | purity (%) | | | pH of the solution | | | of the solution | | |
| weeks | pH 4.9 | pH 4.5 | pH 4.0 | pH 4.9 | pH 4.5 | pH 4.0 | pH 4.9 | pH 4.5 | pH 4.0 |
| 0 | 99.04 | 99.04 | 99.04 | 4.94 | 4.52 | 4.01 | 246 | 299 | 355 |
| 1 | 97.93 | 98.21 | 98.50 | 4.94 | 4.53 | 4.01 | 248 | 295 | 360 |
| 2 | 96.64 | 97.17 | 97.72 | 4.96 | 4.55 | 4.04 | 252 | 300 | 367 |
| 3 | 95.64 | 96.39 | 97.30 | 4.89 | 4.46 | 3.92 | 226 | 264 | 335 |
| 4 | 94.78 | 95.72 | 96.97 | 4.82 | 4.40 | 3.87 | 247 | 297 | 366 |
| 8 | 92.07 | 93.58 | 95.65 | 4.92 | 4.51 | 4.00 | 245 | 293 | 360 |
| 12 | 89.08 | 90.98 | 93.93 | 4.90 | 4.47 | 3.95 | 245 | 297 | 362 |
| 24 | 83.68 | 85.67 | 89.87 | 5.01 | 4.60 | 4.07 | 260 | 310 | 382 |

### Determination of Appropriate Components for the Neutralization Solution

The solution described in Example 1 (pH 4.0) was singled out as starting material. Sodium hydroxide (1N) was chosen as neutralizing agent to reach a final pH in the range of 6.5-7.5. Phosphate buffer was chosen for stabilizing the pH in the range 6.5-7.5 after neutralization. The final concentration of 50 mM of phosphate buffer at pH 7 was selected to reach an osmolality in the range of about 150-500 mOsm/kg suitable for the final solution for nebulization.

### Preparation of the Phosphate Buffer 200 mM at pH 7 (Non-GMP)

2.4 g of sodium phosphate monobasic anhydrous were solubilized in 75 mL of water for injection (WFI) in a 100 mL beaker under magnetic stirring. pH was adjusted to 7 with 14 mL of a solution of NaOH 1N. The solution was transferred in a 100 mL volumetric flask and was completed to 100 mL with WFI.

### Example 4: Preparation of a Solution of the Acetate Salt of Cyclo(-Thr-Trp-Ile-Dab-Orn-^{D}Dab-Dab-Trp-Dab-Dab-Ala-Ser-^{D}Pro-Pro-) at 25 mg/mL Net Peptide Content at pH 7

2000 μL of Example 1 were mixed with 1000 μL phosphate buffer 200 mM pH 7. NaOH 1N solution was added until pH 7 was reached (total volume: 400 μL) and volume was adjusted to 4000 μL using water for injection (600 μL).
Final pH=7.0 (method B)
Osmolality=384 mOsm/kg (method C)

Example 4 demonstrates that the final solution for nebulization has an osmolality within the range of 250-500 mOsm/kg suitable for nebulization.

### Example 5: Neutralization Solution (Non-GMP)

20 mL (20.8 g, d=1.04 g/mL) of a solution of NaOH 1N were mixed with 50 mL (51 g, d=1.02 g/mL) of phosphate buffer 200 mM pH 7 (see above) in a volumetric flask. The volume was adjusted to 100 mL with WFI.
Final pH=13.1 (method B)
Osmolality=341 mOsm/kg (method C)

### Example 6: Final Solution for Nebulization

4 mL of Example 5 were added to 4 mL of Example 1 and stirred 5 min.
Final pH=7.0
Osmolality=363 mOsm/kg

Example 6 demonstrates that the process to obtain the final solution for nebulization by adding a neutralization solution as described in Example 5 to a concentrated solution of the acetate salt of cyclo(-Thr-Trp-Ile-Dab-Orn-^{D}Dab-Dab-Trp-Dab-Dab-Ala-Ser-^{D}Pro-Pro-) (drug substance) as described in Example 1 is feasible and robust as indicated by the two parameters pH and osmolality.

### Stability of Example 6 During Nebulization

8 mL of Example 6 were nebulized at room temperature (25° C.) using an eFlow™ nebuliser, header type class 30 (reservoir of 8 mL, PARI GmbH, Starnberg, Germany) over 20 minutes. The nebulized solution was collected in a tube for 5 min interval (tube changed after each interval) and LC-MS analysis of each tube (method A) was performed to evaluate the purity.

TABLE 3
Stability of the final solution obtained in Example
6 during nebulization over 20 minutes.

| | cyclo(-Thr-Trp-Ile-Dab- |
|---|---|
| | Orn-^{D}Dab-Dab-Trp-Dab- |
| | Dab-Ala-Ser-^{D}Pro-Pro-) |
| Time (min) | purity (%) |
| 0 | 98.7 |
| 3 | 98.7 |
| 6 | 98.6 |
| 9 | 98.6 |
| 12 | 98.7 |
| 15 | 98.6 |
| 20 | 98.5 |

### Manufacture of Drug Product Solutions

### Example 7: Solution of the Acetate Salt of Cyclo(-Thr-Trp-Ile-Dab-Orn-^{D}Dab-Dab-Trp-Dab-Dab-Ala-Ser-^{D}Pro-Pro-) at 50 mg/mL Net Peptide at pH 4 (Solution 1)

345.3 g of the acetate salt of cyclo(-Thr-Trp-Ile-Dab-Orn-^{D}Dab-Dab-Trp-Dab-Dab-Ala-Ser-^{D}Pro-Pro-) (drug substance comprising murepavadin) were dissolved in 3750 g of water for injection. Stirring was continued at least 5 min. The pH of the solution was adjusted to 4.0±0.1 with hydrochloric acid, 1M. WFI was added to final weight of 5074.9 g (=5000 mL) and mixing provided the bulk Drug Product solution. The solution was subjected to a bioburden reduction filtration through a sterilized 0.2 μm membrane filter into a sterilized container. The bulk solution was covered with a nitrogen blanket. In a Class 100/Grade A environment, the murepavadin acetate salt solution was sterile-filtered through a sterilized 0.2 μm membrane filter (redundant set-up of two off-line filters in sequence) into a sterilized container and filled into depyrogenated, sterile 10R (10 mL) Ph. Eur. hydrolytic class I, amber glass vials at a fill weight of 4.272 g per vial, corresponding to a fill volume of 4.2 mL per vial. The fill volume included an excess volume (overfill) of 0.2 mL, to ensure that the nominal/extractable volume of 4.0 mL can be withdrawn and administered. Each vial was stoppered with a sterilized rubber stopper. Each stoppered vial is crimped with an aluminium oversea) and flip-off cap.

The manufacturing steps were conducted at ambient temperature. After visual inspection for defects, the vials were transferred to the 2-8° C. storage. The equipment used for the sterile filtration and filling process as well as the components of the primary packaging were sterilized by wet heat in an autoclave (122° C., 30 min.). The filtration process consisted of (i) a pre-filtration from zone C to B and (ii) filtration in zone A. For the filtration a redundant set-up of 2 off-line filters is used, i.e. the second off-line filter was a precautionary measure and not required for achieving the specifications.

### Example 8: Solution for Neutralization (Solution 2)

a) Preparation of the Phosphate Buffer Solution at 200 mM Concentration and pH 7

Sodium dihydrogen phosphate dihydrate (152.6 g) was dissolved in 3776.5 g of water for injection. Stirring is continued at least 5 min. The pH of the solution was adjusted to 7.0±0.1 with sodium hydroxide solution, 1M. (approximately 714 g). WFI is added to final weight of 5000 g (=5000 mL) and mixing provides the phosphate buffer solution.

b) Preparation of the Final Solution for Neutralization

1976 g of sodium hydroxide solution at 1N were slowly added to 4845 g of phosphate buffer at 200 mM pH 7 under stirring. Stirring is continued at least 5 min. The pH of the solution was adjusted to 13.0±0.1 with sodium hydroxide solution, 1M. (approximately 714 g). WFI is added to final weight of 9614 g (=9500 mL) and mixing provides the phosphate buffer solution.

The solution was subjected to a bioburden reduction filtration through a sterilized 0.2 μm membrane filter into a sterilized container. The bulk solution was covered with a nitrogen blanket. In a Class 100/Grade A environment, the solution was sterile-filtered through a sterilized 0.2 μm membrane filter (redundant set-up of two off-line filters in sequence) into a sterilized container and filled into depyrogenated, sterile 4R (10 mL) Ph. Eur. hydrolytic class I, amber glass vials at a fill weight of 5.101 g per vial, corresponding to a fill volume of 5.0 mL per vial. The fill volume included an excess volume (overfill) of 1 mL, to ensure that the nominal/extractable volume of 4.7 mL can be withdrawn and administered. Each vial was stoppered with a sterilized rubber stopper. Each stoppered vial is crimped with an aluminium oversea) and flip-off cap.

The manufacturing steps were conducted at ambient temperature. After visual inspection for defects, the vials were transferred to the 2-8° C. storage. The equipment used for the sterile filtration and filling process as well as the components of the primary packaging were sterilized by wet heat in an autoclave (122° C., 30 min.). The filtration process consisted of (i) a pre-filtration from zone C to B and (ii) filtration in zone A. For the filtration a redundant set-up of 2 off-line filters is used, i.e. the second off-line filter was a precautionary measure and not required for achieving the specifications.

Further characterization of solution 1 and the final solution for nebulisation

### Example 9: Surface Tension

Surface tension of solution 1, the final solution for nebulisation after addition of 4.2 mL of solution 2 to 4.2 mL of solution 1 and corresponding dilution of final solution for nebulisation with 0.9% NaCl measured using method D (see Table 4):

TABLE 4
Surface tension of solution 1 and final solution
for nebulization at different concentrations

| Tested solution of | |
|---|---|
| cyclo(-Thr-Trp-Ile-Dab-Orn-^{D}Dab-Dab-Trp- | Surface |
| Dab-Dab-Ala-Ser-^{D}Pro-Pro-) and | Tension |
| corresponding concentration thereof | (mN/m) |
| Solution 1 [50 mg/mL] | 66.6 ± 0.1 |
| Final solution for nebulisation [25 mg/mL] | 71.4 ± 0.1 |
| Final solution for nebulisation [1.56 mg/mL] | 68.2 ± 0.1 |

### Example 10: Viscosity

Viscosity of solution 1, the final solution for nebulisation after addition of 4.2 mL of solution 2 to 4.2 mL of solution 1 and corresponding dilution with 0.9% NaCl measured using method E (see Table 5):

TABLE 5
Viscosity of solution 1 and final solution for
nebulization at different concentrations

| Tested solution of | |
|---|---|
| cyclo(-Thr-Trp-Ile-Dab-Orn-^{D}Dab-Dab-Trp- | Dynamic |
| Dab-Dab-Ala-Ser-^{D}Pro-Pro-) and | Viscosity |
| corresponding concentration thereof | (mPa*s) |
| Solution 1 [50 mg/mL] | 1.25 ± 0.02 |
| Final solution for nebulisation [25 mg/mL] | 1.14 ± 0.01 |
| Final solution for nebulisation [2.78 mg/mL] | 0.99 ± 0.02 |

### Example 11: Breath Simulation

Breath simulation of solution 1 was performed following method F after corresponding dilutions of solution 1 with 0.9% NaCl to obtain solutions of 25 mg/mL and 1.56 mg/mL net peptide content, respectively. Total volume used: 8 mL.

TABLE 6
Results of the breath simulation of solution 1 at different concentrations

| | Strength of | | Drug in | | Drug in | | Neb |
|---|---|---|---|---|---|---|---|
| | solution | DD¹⁾ | Residue²⁾ | DD¹⁾ | Residue²⁾ | DDR³⁾ | Time⁴⁾ |
| Device | (mg/mL) | (mg) | (mg) | (%) | (%) | (mg/min) | (min) |
| Device 1 | 25.00 | 124.68 | 31.71 | 58.72 | 14.9 | 4.21 | 20.8 |
| Device 2 | 25.00 | 125.33 | 28.88 | 59.03 | 13.6 | 4.94 | 21.7 |
| Device 3 | 25.00 | 113.34 | 40.00 | 53.32 | 18.8 | 6.00 | 16.8 |
| Device 1 | 25.00 | 125.92 | 25.03 | 59.25 | 11.8 | 4.45 | 24.2 |
| Device 2 | 25.00 | 124.18 | 26.45 | 58.45 | 12.5 | 4.63 | 22.7 |
| Device 3 | 25.00 | 113.74 | 38.74 | 53.50 | 18.2 | 6.03 | 18.6 |
| Mean | | 121.20 | 31.80 | 57.05 | 15.0 | 5.04 | 20.8 |
| SD | | 5.96 | 6.30 | 2.83 | 3.0 | 0.79 | 2.7 |
| % RSD | | 4.9 | 19.8 | 4.96 | 19.8 | 15.64 | 13.0 |
| Device 1 | 1.56 | 7.66 | 1.37 | 58.14 | 10.4 | 0.35 | 21.6 |
| Device 2 | 1.56 | 8.07 | 1.63 | 61.28 | 12.4 | 0.35 | 22.9 |
| Device 3 | 1.56 | 7.50 | 1.93 | 56.90 | 14.6 | 0.43 | 17.6 |
| Device 1 | 1.56 | 7.83 | 1.25 | 59.49 | 9.5 | 0.35 | 22.6 |
| Device 2 | 1.56 | 7.84 | 1.56 | 59.60 | 11.8 | 0.34 | 23.0 |
| Device 3 | 1.56 | 7.74 | 1.71 | 58.78 | 13.0 | 0.37 | 21.1 |
| Mean | | 7.77 | 1.57 | 59.03 | 11.9 | 0.36 | 21.5 |
| SD | | 0.19 | 0.24 | 1.48 | 1.8 | 0.03 | 2.0 |
| % RSD | | 2.5 | 15.4 | 2.5 | 15.4 | 8.5 | 9.5 |

¹⁾DD = Delivered Dose: Total drug delivered in (mg) or (% of filled dose)
²⁾Drug in Residue: Drug mass found in the nebulizer after nebulization in (mg) or (% of loaded dose)
³⁾DDR = Drug Delivery Rate: Aerosol output in 1 minute in (mg/min)
⁴⁾Neb Time: Automatic shut off in (min) or end of aerosol production in (min)

### Example 12: Determination of Aerodynamic Particle Size Distribution (APSD)

Aerodynamic Particle Size Distribution (APSD) was determined using Next Generation Impactor (NGI) after corresponding dilutions of solution 1 with 0.9% NaCl to obtain solutions of 25 mg/mL and 1.56 mg/mL net peptide content, respectively. Total volume used: 8 mL. The NGI experiment was conducted according to USP chapter <1601> respectively Ph. Eur. 2.9.18. Evaporation of droplets produced by nebulizers was minimized by maintaining the impactor temperature to the aerosol temperature of 18±0.5° C.

TABLE 7
Results of Aerodynamic Particle Size Distribution
(APSD) of solution 1 at different concentrations

| | Strength of | | | | | | Mass |
|---|---|---|---|---|---|---|---|
| | solution | MMAD¹⁾ | | FPD³⁾ | FPF⁴⁾ | RD⁵⁾ | Balance⁶⁾ |
| Device | (mg/mL) | (μm) | GSD²⁾ | (mg) | (%) | (mg) | (%) |
| Device 1 | 25.00 | 3.31 | 1.70 | 147.84 | 77.9 | 97.13 | 93.0 |
| Device 2 | 25.00 | 3.28 | 1.69 | 146.26 | 78.8 | 98.70 | 92.0 |
| Device 3 | 25.00 | 3.49 | 1.71 | 151.11 | 75.0 | 85.06 | 94.5 |
| Device 1 | 25.00 | 3.52 | 1.69 | 143.88 | 75.1 | 94.54 | 93.5 |
| Device 2 | 25.00 | 3.47 | 1.70 | 139.48 | 75.5 | 93.75 | 91.0 |
| Device 3 | 25.00 | 3.53 | 1.68 | 143.22 | 75.2 | 85.54 | 93.1 |
| Mean | | 3.43 | 1.69 | 145.30 | 76.2 | 92.45 | 92.9 |
| SD | | 0.11 | 0.01 | 4.03 | 1.6 | 5.82 | 1.2 |
| % RSD | | 3.2 | 0.7 | 2.8 | 2.2 | 6.3 | 1.3 |
| Device 1 | 1.56 | 3.52 | 1.69 | 8.61 | 75.0 | 5.74 | 90.4 |
| Device 2 | 1.56 | 3.64 | 1.76 | 7.87 | 71.1 | 5.73 | 89.4 |
| Device 3 | 1.56 | 3.54 | 1.71 | 8.78 | 74.5 | 5.59 | 92.5 |
| Device 1 | 1.56 | 3.38 | 1.70 | 9.01 | 76.9 | 6.02 | 93.4 |
| Device 2 | 1.56 | 3.41 | 1.72 | 9.39 | 76.2 | 5.98 | 96.3 |
| Device 3 | 1.56 | 3.36 | 1.68 | 8.53 | 78.1 | 6.05 | 88.8 |
| Mean | | 3.48 | 1.71 | 8.70 | 75.3 | 5.85 | 91.8 |
| SD | | 0.11 | 0.03 | 0.51 | 2.5 | 0.19 | 2.8 |
| % RSD | | 3.2 | 1.7 | 5.9 | 3.3 | 3.2 | 3.1 |

¹⁾MMAD = Mass Median Aerodynamic Diameter (μm)
²⁾GSD = Geometric Standard Deviation
³⁾FPD = Fine particle dose <5 μm
⁴⁾FPF = Fine particle fraction <5 μm
⁵⁾RD = Respirable dose (fine particle mass in mg): Part of the DD (see Example 11) contained in droplets <5 μm (DD × FPF)
⁶⁾Mass Balance was calculated using the actual content of the formulation

From the results presented in Examples 11 and 12 above, no significant changes were seen in the performance of the solutions of different strength within the presented range.

### Example 13: Stability of Drug Product Solutions (Example 7) Over Time

Two drug product solutions Example 13a and 13b (solutions of the acetate salt of cyclo(-Thr-Trp-Ile-Dab-Orn-^{D}Dab-Dab-Trp-Dab-Dab-Ala-Ser-^{D}Pro-Pro-) at 50 mg/mL net peptide at pH 4) were prepared for stability studies. The study design is in accordance with the recommendations of the ICH guideline Q1A, version Q1A(R2), and Q1B. Table 8 shows different dimensions establishing the stability of the two drug product solutions.

TABLE 8
Stability results based on measurements of osmolality (method C),
pH (method B), purity (method A), surface tension (method D) and
dynamic viscosity (method E) of two drug product solutions

| | | | Storage Time | | |
|---|---|---|---|---|---|
| Storage | | | [months] | | |
| Conditions | Batch | Dimension | 0 | 1 | 3 |

Osmolality
(mOsm/kg)

| | | | | |
|---|---|---|---|---|
| 5 ± 3° C. | Example 7a | 363 | 365 | 365 |
| 5 ± 3° C. | Example 7b | 363 | 362 | 352 |

pH

| | | | | |
|---|---|---|---|---|
| 5 ± 3° C. | Example 7a | 4.0 | 4.1 | 4.1 |
| 5 ± 3° C. | Example 7b | 4.1 | 4.1 | 4.1 |

Purity
(%)

| | | | | |
|---|---|---|---|---|
| 5 ± 3° C. | Example 7a | 99 | 98.8 | 98.3 |
| 5 ± 3° C. | Example 7b | 98.9 | 98.6 | 98.8 |

Surface tension
(mN/m)

| | | | | |
|---|---|---|---|---|
| 5 ± 3° C. | Example 7a | 66.4 | 66.5 | 66.3 |
| 5 ± 3° C. | Example 7b | 67 | 66.5 | 66.7 |

Dynamic Viscosity
(mPas*s)

| 5 ± 3° C. | Example 7a | | 1.26 | 1.22 | 1.21 |
|---|---|---|---|---|---|
| 5 ± 3° C. | Example 7b | | 1.24 | 1.21 | 1.26 |
| | | | Storage Time | | |
| Storage | | | [months] | | |
| Conditions | Batch | Dimension | 6 | 9 | 12 |

Osmolality
(mOsm/kg)

| | | | | |
|---|---|---|---|---|
| 5 ± 3° C. | Example 7a | 361 | 367 | 367 |
| 5 ± 3° C. | Example 7b | 358 | 359 | 359 |

pH

| | | | | |
|---|---|---|---|---|
| 5 ± 3° C. | Example 7a | 4.1 | 4.1 | 4.0 |
| 5 ± 3° C. | Example 7b | 4.2 | 4.1 | 4.1 |

Purity
(%)

| | | | | |
|---|---|---|---|---|
| 5 ± 3° C. | Example 7a | 98.2 | 97.9 | 97.5 |
| 5 ± 3° C. | Example 7b | 98.3 | 98.3 | 97.7 |

Surface tension
(mN/m)

| | | | | |
|---|---|---|---|---|
| 5 ± 3° C. | Example 7a | 66.7 | 66.5 | 66.8 |
| 5 ± 3° C. | Example 7b | 66.7 | 66.6 | 66.9 |

Dynamic Viscosity
(mPas*s)

| | | | | |
|---|---|---|---|---|
| 5 ± 3° C. | Example 7a | 1.27 | 1.26 | 1.25 |
| 5 ± 3° C. | Example 7b | 1.26 | 1.31 | 1.30 |

The physicochemical properties as well as the purity of the two drug product solutions remain nearly unchanged during 12 months at 5° C.

### Example 14: Stability of Final Solution for Nebulization Prepared from Solutions 1 and 2 Presented in Examples 7 and 8, Respectively

4.2 mL of solution 2 (Example 8) were transferred into a vial containing 4.2 mL of solution 1 (Example 7) and mixed well. This operation was performed 11 times using 11 different active vials. The contents of the 11 vials were then pooled and mixed well. Aliquots of 7.8 mL were transferred to the reservoir of an eFlow™ nebuliser, header type class 30 (reservoir of 8 mL, PARI GmbH, Starnberg, Germany). Samples having a concentration of 25 mg/mL were stored at room temperature (25±2° C.).

TABLE 9
Stability of the final solution for nebulization
(25 mg/mL) at 25 ± 2° C.

| | cyclo(-Thr-Trp-Ile-Dab- |
|---|---|
| | Orn-^{D}Dab-Dab-Trp-Dab- |
| | Dab-Ala-Ser-^{D}Pro-Pro-) |
| Time (min) | purity (%) |
| 0 | 98.7 |
| 60 | 98.5 |

### Example 15: Establishment of a Dose in Human for Clinical Trial

The starting dose of murepavadin inhaled solution (MIS) in this study took both toxicology findings and PK-PD data into account.

In the 28-day dose inhalation study in CD-1 mice (20-minute snout only exposure), the mean estimated achieved doses of 0.8, 3.5 and 7.2 mg/kg/day murepavadin free base were administered. Based on the results of this study, there was no evidence of systemic toxicity up to 7.2 mg/kg/day murepavadin free base, however, due to adverse pathology in the upper respiratory tract, no No Observed Adverse Effect Level (NOAEL) could be established. Following inhalation administration of murepavadin to cynomolgus monkeys (20-minute exposure via face mask) for 4 weeks, at mean estimated achieved doses of 1.5, 4.0 and 10.7 mg/kg/day murepavadin free base, microscopic test item related findings were reported at the tracheal bifurcation of both sexes given 10.7 and 4.0 mg/kg/day, and also in one female given 1.5 mg/kg/day, but were not considered adverse and were not present in the recovery animals. There were no other findings considered test item related.

The efficacious human exposure was estimated based on a mouse lung infection model. In this model, murepavadin was administered by subcutaneous injection. A mean AUC exposure in ELF of 12.23 h·mg/L was sufficient to achieve a 1-login reduction of bacterial burden in 15 clinical Pseudomonas aeruginosa isolates tested. In mouse, 0.10 and 0.18 mg/kg achieved dose were required to attain this ELF AUC exposure by intra-tracheal (i.t.) or inhalation administration, respectively. IT doses of 0.075-0.625 mg/kg even resulted in a >2-log₁₀ reduction of bacterial burden in three out of four clinical Pseudomonas aeruginosa isolates tested in the mouse lung infection model. Accordingly, an achieved dose of 7.0-12.5 mg in a 70 kg human subject could be the pivotal starting point for testing in human. Assuming 50% inhalation efficacy in human this would translate into a dose of 15-25 mg.

### Example 16: Investigational Medicinal Product(s) and Medical Devices to be Used in Clinical Trial

Investigational Medicinal Product is defined as any investigational product undergoing trial(s), marketed product(s), placebo, or medical device(s) intended to be administered to a study participant according to the study protocol.

TABLE 10
Investigational Medicinal Product(s) administered

| | ARM Name | Active | Placebo |
|---|---|---|---|
| | Intervention Name | Murepavadin acetate | Placebo |
| | Type | Drug | Placebo comparator |
| | Route of Administration | Inhalation | Inhalation |
| | Dose Formulation | Nebulizer solution | Nebulizer solution |
| | Inhaled solution | Two-component | Two-component |
| | | preparation, adjusted | preparation, adjusted |
| | | to approx. pH 7.0 | to approx. pH 7.0 |
| | Component 1 | 50 mg/mL (net peptide) | Acetic acid and |
| | (solution 1): | 4.0 | NaCl 0.9% solution |
| | Unit Dose Strength(s) | | 2.8(adjustment with 1M HCl) |
| | pH | | |
| | Component 2 | 100 mM phosphate buffer solution; pH 13 | |
| | (solution 2): | | |
| | Solution for | | |
| | pH adjustment | | |
| Part A | Dose Level 1 | 12.5 mg MPV in 8 mL | Placebo 8 mL |
| | | (1.5 mg/mL) | |
| | Dose Level 2 | 25 mg MPV in 8 mL | Placebo 8 mL |
| | | (3.1 mg/mL) | |
| | Dose Level 3 | 50 mg MPV in 8 mL | Placebo 8 mL |
| | | (6.2 mg/mL) | |
| Part B | Dose Level 4 | 75 mg MPV in 8 mL | Placebo 8 mL |
| | | (9.4 mg/mL) | |
| | Dose Level 5 | 150 mg MPV in 8 mL | Placebo 8 mL |
| | | (18.8 mg/mL) | |
| Part C | Maximum Daily Dose | 400 mg MPV in 16 (2 × 8) | Placebo 16 (2 × 8) mL |
| | Level | mL (25 mg/mL), q.d., | Placebo 8 mL b.i.d. |
| | | OR | |
| | | 200 mg MPV in 8 mL | |
| | | (25 mg/mL) b.i.d. | |
| | Packaging and Labeling | Rubber stoppered vials | Rubber stoppered vials |
| | | containing 4 mL solution. | containing 4 mL solution. |
| | | Each vial will be labelled | Each vial will be labelled as |
| | | as required per country | required per country |
| | | requirement. | requirement. |

MPV: murepavadin = (cyclo(-Thr-Trp-Ile-Dab-Orn-^{D}Dab-Dab-Trp-Dab-Dab-Ala-Ser-^{D}Pro-Pro-)
q.d.: every day/daily
b.i.d.: twice a day

In this trial, murepavadin and placebo will be administered using the eFlow™ Nebulizer controller and handset which creates an aerosol with high density of murepavadin and a defined droplet size to reach the respiratory tract. The eFlow™ Nebulizer Handset, which is part of the eFlow™ device system, is manufactured by PARI Pharma GmbH (Starnberg, Germany). The three (3) main components of the eFlow™ device system are the controller, connection cord, and the eFlow™ Nebulizer Handset.

The investigational medicinal product (IMP) will consist of the:
solution of murepavadin acetate salt at pH4 (50 mg/mL net peptide) and/or placebo solution
solution of phosphate buffer at pH13.1 for the neutralization of the acidic murepavadin solution.

The final nebulized solution is obtained following two (2) successive dilution steps: (i) a 1:1 dilution of the murepavadin (or placebo) solution with the basic solution phosphate buffer solution, and (ii) with NaCl 0.9% to obtain the different doses to be delivered. Depending on the dose, step 2 may not be required.

The final placebo solution for inhalation is prepared to match the frequency and volume of the respective MIS dosing regimen. Depending on the dose, step 2 may not be required.

The final solution will be delivered through a nebulizer handset based on eFlow™ technology (PARI Pharma GmbH, Starnberg, Germany).

### Example 17: Determination of Activities of Murepavadin and Comparators Tobramycin, Colistin and Aztreonam, Against Cystic Fibrosis (CF) Pseudomonas aeruginosa Isolates Growing in Biofilms

Two types of assays, an open and a closed system, were used to evaluate the in vitro activity on biofilms. The closed or static system based on the Calgary device (Diez-Aguilar M et al., Antimicrob Agents Chemother. 2018, 62, e01650-17, doi: 10.1128/AAC.01650-17, Moskowitz S M et al., J. Clin. Microbiol. 2004, 42, 1915-1922) analyzes biofilm formation in the wells of microtiter plates and is suitable for high-throughput analysis, obtaining pharmacodynamics (PD) parameters, while the open or dynamic system using the BioFlux device (Benoit M R et al., Appl. Environ. Microbiol. 2010, 76, 4136-4142) better resembles the in vivo conditions.

### Strains

To perform biofilm tests with the Calgary device, fifty-three Pseudomonas aeruginosa isolates from respiratory samples of people with cystic fibrosis were selected among the 414 Pseudomonas aeruginosa of the iABC-collection (Ekkelenkamp M B et al., Antimicrob. Agents Chemother. 2019, 64, 1-7). A representative number of isolates from Northern Ireland, Spain, the Netherlands, and Australia were chosen, including different morphotyes: 18 smooth, 11 mucoid, 11 rough, 10 metallic, and 3 small colony variants. As control strains, the PAO (most commonly used strain for research on this ubiquitous and metabolically versatile opportunistic pathogen, Klockgether J et al., Journal of Bacteriology 2010, 192, 1113-1121) and its hypermutator derivative, PAO mutS strains were used (Oliver A et al., Antimicrob. Agents Chemother. 2004, 48, 4226-33).

Two CF isolates, PA34 (smooth phenotype) and PA40 (mucoid phenotype), and the 2 reference strains PAO and PAO mutS were tested with the BioFlux device. None of the clinical strains were hypermutators.

### Biofilm Assays Performed by the Calgary Device

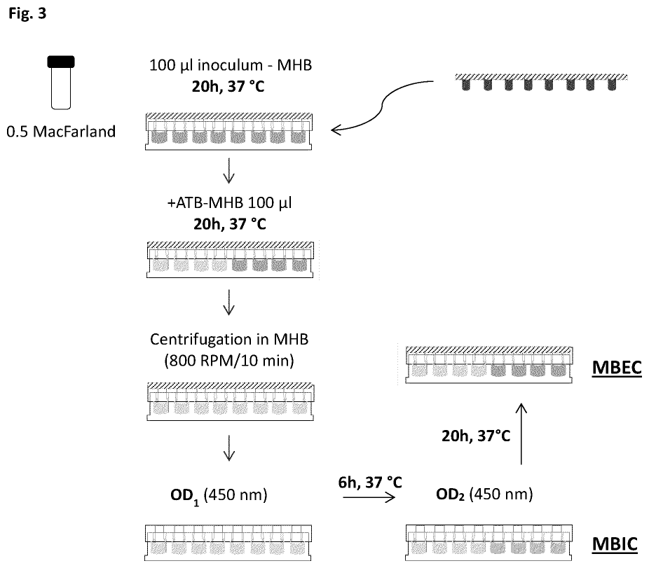

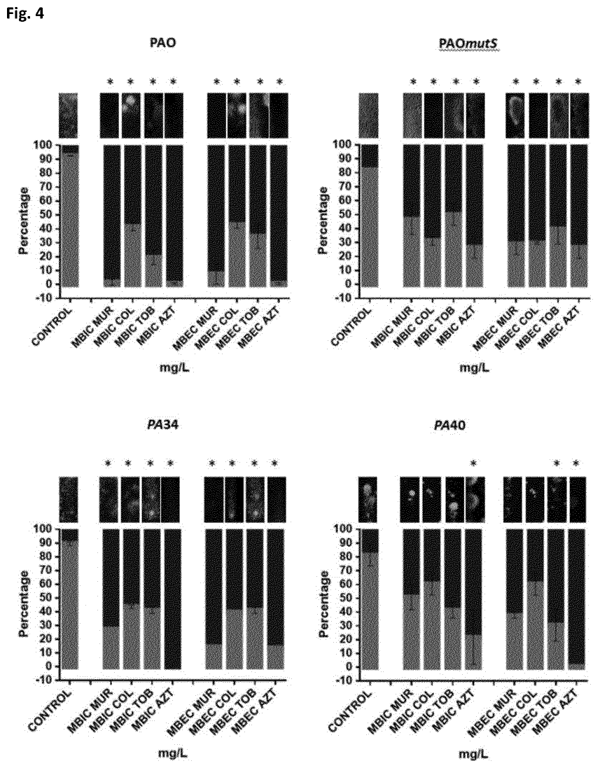

This assay was performed as previously described but with minimal variations (Benoit M R et al., Appl. Environ. Microbiol. 2010, 76, 4136-4142) (FIG. 3). Briefly, a 0.5 McFarland culture was transferred to a flat-bottom 96-well microtiter plate (Nalge Nunc International, Rochester, New York, USA). Bacterial biofilm was formed around the pegs of a modified polystyrene microtiter lid. This lid with pegs was immersed into a growth plate and incubated for 20 h at 37° C. After rinsing the pegs 3 times in saline solution they were placed onto the antimicrobial in BBL™ Mueller-Hinton II cation-adjusted broth (MHB) (Becton, Dickinson and Company, Sparks, MD, USA) and incubated for 20 h at 37° C. Two-fold increasing (0.5-512 mg/L) colistin, tobramycin, aztreonam, and murepavadin (antibiotics [ATB]) concentrations were used. After this incubation, the biofilm was recovered centrifuging (800 RPM/10 min) the peg lid in an antibiotic-free MHB filled-microtiter plate. The minimal biofilm inhibitory concentration (MBIC) was calculated after measuring the optical density (OD) (450 nm) before and after 6-hour incubation. Biofilm growth was defined as a mean OD difference of ≥0.05. MBIC was defined as the lowest antibiotic concentration that resulted in an OD difference at or below 10% of the OD positive control. The minimal biofilm eradication concentration (MBEC) was defined as the lowest concentration that prevents visible growth in the biofilm recovery medium after 18 h of the microtiter plate incubation (Ceri H et al., J. Clin. Microbiol. 1999, 37, 1771; Macià M D et al., Enferm. Infecc. Microbiol. Clin. 2018, 36, 375-381).

Thirty-two (60.4%) isolates used in the above assay were biofilm producers. Out of these 32 isolates, 8 of them were weak producer (<25 percentile), 16 moderate (25-75 percentile) and 8 strong (>75 percentile). MIC_{50/90}, MBIC_{50/90}, and MBEC_{50/90}, and MIC, MBIC and MBEC range results for each antibiotic are described in Table 11.

TABLE 11
Planktonic (MIC₅₀/MIC₉₀) and biofilm (MBIC₅₀/MBIC₉₀, MBEC₅₀/MBEC₉₀) activities for each
tested antibiotic, MUR (murepavadin), COL (colistin), TOB (tobramycin), AZT (aztreonam).

| mg/L | MIC₅₀/MIC₉₀ | MIC range | MBIC₅₀/MBIC₉₀ | MBIC range | MBEC₅₀/MBEC₉₀ | MBEC range |
|---|---|---|---|---|---|---|
| MUR | ≤0.5/1 | ≤0.5-32 | 4/32 | ≤0.5-64 | 16/64 | 1->512 |
| COL | 1/4 | ≤0.5-16 | 8/64 | ≤0.5-256 | 64/128 | ≤0.5-256 |
| TOB | 2/16 | ≤0.5-256 | 2/32 | ≤0.5-512 | 8/256 | ≤0.5-512 |
| AZT | 4/256 | 0.5-512 | 32/>512 | 1->512 | 512/>512 | 1->512 |

MBIC and the MBEC are used to determine the anti-biofilm antibiotic in vitro efficacy. MBIC is the lowest concentration at which there is no time-dependent increase in the mean number of biofilm viable cells comparing an early with a later exposure time, while the MBEC indicates the eradication (partly or completely) of biofilm viable cells (Thieme L et al., Biol. Proced. Online 2019, 21, 1-5).

While murepavadin and colistin presented the best activity against planktonic cells (≤0.5/1 mg/L, 1/4 mg/L, respectively), murepavadin and tobramycin showed the best efficacy against biofilm growth (4/32 mg/L, 2/32 mg/L, respectively). Murepavadin exhibited the lowest MBEC 90 among the tested antibiotics (64 mg/L).

### Biofilm Assays Performed by the BioFlux Microfluidic Open System

Several biofilms were simultaneously developed in a 48 wells plate in the BioFlux 200 system, following an adapted protocol from Benoit M R et al. (Appl. Environ. Microbiol. 2010, 76, 4136-4142). Microchannels were filled with 100 μL of prewarmed diluted (0.1×) Luria Broth media (LB) (Oxoid Ltd., Basingstoke, Hampshire, UK) through the input wells (5 min, 1 dyne/cm²). For the cell attachment, 85 μl of a 108-109 CFU/mL bacterial suspension were inoculated into the output wells for 5 seconds at 2 dyne/cm² and incubated without flow for 2 h at 30° C. After these 2 h, biofilm was formed along 18 h of continuous flow at 30° C. (0.15 dyne/cm²). Biofilm formation was checked after the incubation and antibiotic was added to the inlet wells, (except for the positive control), incubating the biofilm under continuous flow for 18 h (30° C., 0.15 dyne/cm²). The tested antibiotic concentrations corresponded to those of antibiotic MBIC and MBEC values obtained with the Calgary device. Tests were performed in triplicate. After incubation, biofilms were washed by injecting saline solution from the input reservoir for 10 min at 0.5 dynes. The biofilm was stained with the BacLight LIVE/DEAD stain (Invitrogen, ThermoFisher Scientific, Paisley, UK) which consists of SYTO™ 9 and propidium iodide (PI) prepared according to the manufacturer's instructions. These stains were pumped through the channels (0.7 dynes, 15 min) and afterwards the channels were washed 20 min with saline solution to remove excess of stain. Biofilm images were taken using a Nikon Eclipse Ti microscope (Nikon, Tokyo, Japan) and analyzed with the ImageJ program (Java-based image processing program, National Institute of Health, Bethesda, Maryland, USA and Laboratory for Optical and Computational Instrumentation, University of Wisconsin, Wisconsin, USA). The mean percentage of intensity of red fluorescence corresponding to dead cells within the biofilm is represented in a bar chart as black bar (FIG. 4). The mean percentage of intensity of green fluorescence corresponding to live cells within the biofilm is represented in a bar chart as grey bar (FIG. 4). The results of untreated and treated biofilms were examined with ANOVA (analysis of variance) statistical analysis applying the Scheffé correction, considering values of p<0.0125 significantly different. Stata™ statistical software was used (Data Analysis and Statistical Software version 11.0, StataCorp LLC, Texas, USA).

Planktonic and biofilm susceptibility of the strains established in the Calgary device and used in the BioFlux device are described in Table 12.

TABLE 12
Susceptibility of the strains used in the BioFlux device

| ANTIBIOTIC | (mg/L) | PAO | PAO mutS | PA40 | PA34 |
|---|---|---|---|---|---|
| Murepavadin | MIC | 0.06 | 0.12 | 0.06 | 0.06 |
| | MBIC | 4 | 32 | 2 | 2 |
| | MBEC | 8 | 256 | 8 | 4 |
| Aztreonam | MIC | 4 | 64 | 16 | 0.5 |
| | MBIC | >256 | >256 | 32 | 8 |
| | MBEC | >256 | >256 | >256 | 256 |
| Tobramycin | MIC | 0.5 | 2 | 0.5 | 0.25 |
| | MBIC | 2 | 16 | 2 | 2 |
| | MBEC | 16 | 32 | 4 | 2 |
| Colistin | MIC | 1 | 1 | 0.5 | 2 |
| | MBIC | 64 | 256 | 32 | 32 |
| | MBEC | 128 | 256 | 32 | 128 |

All tested strains were susceptible to murepavadin (MIC 0.12 mg/L), colistin (MIC 2 mg/L) and tobramycin (MIC 2 mg/L), showing the best values for murepavadin.

Bar charts are used to describe the BioFlux results in FIG. 4. For the control strains PAO and PAO mutS as well as the PA34 clinical strain a statistically significant difference was observed (p<0.0125, indicated with asterisks in FIG. 4) when comparing the fluorescence registered in the control channel (without antibiotic) with those treated with the antibiotics at every tested concentration (MBIC and MBEC).

Antibiotics' MBICs and MBECs confirmed their efficiency to reduce the biofilm developed in the continuous fluid system for the control strains PAO and PAO mutS as well as the smooth phenotype (PA34) strain.

Murepavadin displayed activity against biofilms of Pseudomonas aeruginosa by both open and closed biofilm-testing systems. Murepavadin, colistin and tobramycin presented similar MBIC₅₀/MBIC₉₀ values (4/32 mg/L, 8/64 mg/L, 2/32 mg/L, respectively) but murepavadin exhibited the lowest MBEC₉₀ among the tested antibiotics (64 mg/L).

1. A liquid composition comprising from about 40 mg/mL to about 60 mg/mL of the active compound cyclo(-Thr-Trp-Ile-Dab-Orn-^{D}Dab-Dab-Trp-Dab-Dab-Ala-Ser-^{D}Pro-Pro-), wherein the composition has a pH from about 3.5 to about 4.5.

2. The liquid composition according to claim 1, wherein the liquid composition is an aqueous composition.

3. The aqueous composition according to claim 2, wherein the acetate salt of the active compound cyclo(-Thr-Trp-Ile-Dab-Orn-^{D}Dab-Dab-Trp-Dab-Dab-Ala-Ser-^{D}Pro-Pro-) is dissolved in the aqueous composition.

4. The composition according to any one of claims 1 to 3, wherein the composition has a pH of about 4.0.

5. The composition according to any one of claims 1 to 4, wherein the composition comprises about 50 mg/mL of the active compound cyclo(-Thr-Trp-Ile-Dab-Orn-^{D}Dab-Dab-Trp-Dab-Dab-Ala-Ser-^{D}Pro-Pro-).

6. A kit comprising:
(a) a first kit component comprising the composition of any one of claims 1 to 5; and
(b) a second kit component comprising a diluent.

7. The kit according to claim 6, wherein the diluent is an aqueous diluent.

8. The kit according to claim 7, wherein the aqueous diluent comprises at least one buffering agent.

9. The kit according to claim 8, wherein the at least one buffering agent is adapted to maintain a pH from about 6.5 to about 7.5, preferably a pH about 7.0, upon combining the first and the second kit components.

10. The kit according to any one of claims 7 to 9, wherein the aqueous diluent comprises a phosphate buffer and has a pH from about 11.0 to about 13.5.

11. A dilution of the composition according to any one of claims 1 to 5, comprising from about 20 mg/mL to about 30 mg/mL of the active compound cyclo(-Thr-Trp-Ile-Dab-Orn-^{D}Dab-Dab-Trp-Dab-Dab-Ala-Ser-^{D}Pro-Pro-), wherein the dilution has a pH from about 6.5 to about 7.5, preferably a pH of about 7.0 and optionally a osmolality from about 150 to about 500 mOsm/kg.

12. The dilution according to claim 11, wherein the diluent is an aqueous diluent comprising at least one buffering agent.

13. The dilution according to claim 12, wherein the at least one buffering agent is adapted to maintain a pH from about 6.5 to about 7.5, preferably a pH about 7.0.

14. The dilution according to any one of claims 12 to 13, wherein the aqueous diluent comprises a phosphate buffer and has a pH from about 11.0 to about 13.5.

15. A pharmaceutical composition comprising the active compound cyclo(-Thr-Trp-Ile-Dab-Orn-^{D}Dab-Dab-Trp-Dab-Dab-Ala-Ser-^{D}Pro-Pro-);
or any pharmaceutically acceptable salt thereof;
and optionally one or more pharmaceutically acceptable diluents, excipients or carriers;
wherein the pharmaceutical composition comprises from about 1.5 mg/mL to about 25 mg/mL of the active compound cyclo(-Thr-Trp-Ile-Dab-Orn-^{D}Dab-Dab-Trp-Dab-Dab-Ala-Ser-^{D}Pro-Pro-).

16. The pharmaceutical composition according to claim 15, wherein the pharmaceutical composition comprises the dilution according to any one of claims 11 to 14, wherein the dilution is further diluted to obtain a pharmaceutical composition comprising about 1.5 mg/mL to about 25 mg/mL of the active compound cyclo(-Thr-Trp-Ile-Dab-Orn-^{D}Dab-Dab-Trp-Dab-Dab-Ala-Ser-^{D}Pro-Pro-).

17. The pharmaceutical composition according to claim 16, wherein the dilution is further diluted by adding an aqueous solution.

18. The pharmaceutical composition according to any one of claims 15 to 17, wherein the pharmaceutical composition has an osmolality from about 150 to about 500 mOsm/kg.

19. The pharmaceutical composition according to any one of claims 15 to 18 for use in a method for the prevention, management or treatment of diseases or conditions of the lungs in a subject.

20. The pharmaceutical composition according to any one of claims 15 to 18, for use in a method for the prevention, management or treatment of biofilm-associated diseases or conditions of the lungs in a subject.

21. The pharmaceutical composition according to any one of claims 15 to 18, for use in a method for eradicating a biofilm in biofilm-associated diseases or conditions of the lungs in a subject.

22. The pharmaceutical composition according to any one of claims 15 to 18, for use in a method for eradicating a biofilm in biofilm-associated diseases or conditions.

23. The pharmaceutical composition according to any one of claims 15 to 18, for use in a method for the prevention, management or treatment of biofilm formation.

24. A pharmaceutical composition for use according to any one of claims 19 to 21, wherein the diseases or conditions of the lungs are selected from the group consisting of cystic fibrosis (CF), non-cystic fibrosis bronchiectasis (NCFB), asthma, chronic obstructive pulmonary disease (COPD), ventilator-associated pneumonia (VAP), ventilator-associated bacterial pneumonia (VABP), hospital-acquired pneumonia (HAP), hospital-acquired bacterial pneumonia (HABP), and healthcare-associated pneumonia (HCAP).

25. A pharmaceutical composition for use according to any one of claims 19 to 21, wherein the diseases or conditions of the lungs are cystic fibrosis (CF), or non-cystic fibrosis bronchiectasis (NCFB).

26. A pharmaceutical composition for use according to any one of claims 19 to 21, wherein the disease or condition of the lungs is cystic fibrosis (CF).

27. A pharmaceutical composition for use according to any one of claims 19 to 26, wherein the active compound cyclo(-Thr-Trp-Ile-Dab-Orn-^{D}Dab-Dab-Trp-Dab-Dab-Ala-Ser-^{D}Pro-Pro-);
or any pharmaceutically acceptable salt thereof; is administered to the subject as pharmaceutical aerosol for pulmonary administration comprising a dispersed liquid phase and a continuous gas phase, wherein the dispersed liquid phase
(a) comprises aqueous droplets comprising the active compound cyclo(-Thr-Trp-Ile-Dab-Orn-^{D}Dab-Dab-Trp-Dab-Dab-Ala-Ser-^{D}Pro-Pro-);
or any pharmaceutically acceptable salt thereof;
(b) has a mass median aerodynamic diameter from about 1.5 μm to about 5 μm; and
(c) has a droplet size distribution having a geometrical standard deviation from about 1.2 to about 1.8.

28. A pharmaceutical composition for use according to any one of claims 19 to 27, wherein the active compound cyclo(-Thr-Trp-Ile-Dab-Orn-^{D}Dab-Dab-Trp-Dab-Dab-Ala-Ser-^{D}Pro-Pro-);
or any pharmaceutically acceptable salt thereof; is administered to the subject at a dose between about 5 and about 500 mg/day of the active compound cyclo(-Thr-Trp-Ile-Dab-Orn-^{D}Dab-Dab-Trp-Dab-Dab-Ala-Ser-^{D}Pro-Pro-).

29. A pharmaceutical composition for use according to any one of claims 19 to 28, wherein the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof is administered to the subject by oral inhalation.

30. A pharmaceutical aerosol for pulmonary administration comprising a dispersed liquid phase and a continuous gas phase, wherein the dispersed liquid phase
(a) comprises aqueous droplets comprising the active compound cyclo(-Thr-Trp-Ile-Dab-Orn-^{D}Dab-Dab-Trp-Dab-Dab-Ala-Ser-^{D}Pro-Pro-);
or any pharmaceutically acceptable salt thereof;
(b) has a mass median aerodynamic diameter from about 1.5 μm to about 5 μm; and
(c) has a droplet size distribution having a geometrical standard deviation from about 1.2 to about 1.8.

31. A pharmaceutical aerosol for pulmonary administration according to claim 30, wherein the aerosol comprises from about 1.5 mg/mL to about 25 mg/mL of the active compound cyclo(-Thr-Trp-Ile-Dab-Orn-^{D}Dab-Dab-Trp-Dab-Dab-Ala-Ser-^{D}Pro-Pro-).

32. The pharmaceutical aerosol for pulmonary administration according to claim 30 or 31, wherein the dispersed liquid phase of the aerosol has an osmolality from about 150 to about 500 mOsm/kg.

33. A pharmaceutical aerosol for pulmonary administration according to any one of claims 30 to 32, for use in a method for the prevention, management or treatment of diseases or conditions of the lungs in a subject.

34. A pharmaceutical aerosol for pulmonary administration according to any one of claims 30 to 32, for use in a method for the prevention, management or treatment of biofilm-associated diseases or conditions of the lungs in a subject.

35. A pharmaceutical aerosol for pulmonary administration according to any one of claims 30 to 32, for use in a method for eradicating a biofilm in biofilm-associated diseases or conditions of the lungs in a subject.

36. A pharmaceutical aerosol for pulmonary administration according to any one of claims 30 to 32, for use in a method for eradicating a biofilm in biofilm-associated diseases or conditions.

37. A pharmaceutical aerosol for pulmonary administration according to any one of claims 30 to 2, for use in a method for the prevention, management or treatment of biofilm formation.

38. A pharmaceutical aerosol for use according to any one of claims 33 to 35, wherein the diseases or conditions of the lungs are selected from the group consisting of cystic fibrosis (CF), non-cystic fibrosis bronchiectasis (NCFB), asthma, chronic obstructive pulmonary disease (COPD), ventilator-associated pneumonia (VAP), ventilator-associated bacterial pneumonia (VABP), hospital-acquired pneumonia (HAP), hospital-acquired bacterial pneumonia (HABP), and healthcare-associated pneumonia (HCAP).

39. A pharmaceutical aerosol for use according to any one of claims 33 to 35, wherein the diseases or conditions of the lungs are cystic fibrosis (CF), or non-cystic fibrosis bronchiectasis (NCFB).

40. A pharmaceutical aerosol for use according to any one of claim 33 to 35, wherein the disease or condition of the lungs is cystic fibrosis (CF).

41. A pharmaceutical aerosol for use according to any one of claims 33 to 40, wherein the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof; is administered to the subject at a dose between about 5 and about 500 mg/day of the active compound cyclo(-Thr-Trp-Ile-Dab-Orn-^{D}Dab-Dab-Trp-Dab-Dab-Ala-Ser-^{D}Pro-Pro-).

42. A pharmaceutical aerosol for use according to any one of claims 33 to 41, wherein the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof; is administered to the subject at a dose between about 0.03 and about 7.2 mg/kg of the active compound cyclo(-Thr-Trp-Ile-Dab-Orn-^{D}Dab-Dab-Trp-Dab-Dab-Ala-Ser-^{D}Pro-Pro-).

43. A pharmaceutical aerosol for use according to any one of claims 33 to 42, wherein the active compound cyclo(-Thr-Trp-Ile-Dab-Orn-^{D}Dab-Dab-Trp-Dab-Dab-Ala-Ser-^{D}Pro-Pro-); or any pharmaceutically acceptable salt thereof; is administered to the subject by oral inhalation.

44. A kit for the preparation and delivery of a pharmaceutical aerosol for pulmonary administration comprising a dispersed liquid phase and a continuous gas phase, wherein the dispersed liquid phase
(a) comprises aqueous droplets comprising the active compound cyclo(-Thr-Trp-Ile-Dab-Orn-^{D}Dab-Dab-Trp-Dab-Dab-Ala-Ser-^{D}Pro-Pro-);
or any pharmaceutically acceptable salt thereof;
(b) has a mass median aerodynamic diameter from about 1.5 μm to about 5 μm; and
(c) has a droplet size distribution having a geometrical standard deviation from about 1.2 to about 1.8;
and wherein the kit comprises a nebulizer and a pharmaceutical composition comprising a concentration within a range from about 1.5 mg/mL to about 25 mg/mL of the active compound.

45. A kit according to claim 44, wherein the liquid composition of the aerosol has an osmolality from about 150 to about 500 mOsm/kg.

46. A kit according to claim 44 or 45, wherein the nebulizer is selected from the group consisting of jet nebulizers, ultrasonic nebulizers, piezoelectric nebulizers, jet collision nebulizers, electrohydrodynamic nebulizers, capillary force nebulizers, perforated membrane nebulizers and perforated vibrating membrane nebulizers.

47. A kit according to any one of claims 44 to 46, wherein the kit further comprises a package insert wherein the package insert comprises instructions for treating a subject for diseases or conditions of the lungs.

48. A kit according to any one of claims 44 to 47, for use in a method for the prevention, management or treatment of diseases or conditions of the lungs in a subject.

49. A kit according to any one of claims 44 to 47, for use in a method for the prevention, management or treatment of biofilm-associated diseases or conditions of the lungs in a subject.

50. A kit according to any one of claims 44 to 47, for use in a method for eradicating a biofilm in biofilm-associated diseases or conditions of the lungs in a subject.

51. A kit according to any one of claims 44 to 47, for use in a method for eradicating a biofilm in biofilm-associated diseases or conditions in a subject.

52. A kit according to any one of claims 44 to 47, for use in a method for the prevention, management or treatment of biofilm formation in a subject.

53. A kit for use according to any one of claims 48 to 50, wherein the diseases or conditions of the lungs are selected from the group consisting of cystic fibrosis (CF), non-cystic fibrosis bronchiectasis (NCFB), asthma, chronic obstructive pulmonary disease (COPD), ventilator-associated pneumonia (VAP), ventilator-associated bacterial pneumonia (VABP), hospital-acquired pneumonia (HAP), hospital-acquired bacterial pneumonia (HABP), and healthcare-associated pneumonia (HCAP).

54. A kit for use according to any one of claims 48 to 50, wherein the diseases or conditions of the lungs are cystic fibrosis (CF), or non-cystic fibrosis bronchiectasis (NCFB).

55. A kit for use according to any one of claims 48 to 50, wherein the pulmonary disease is cystic fibrosis (CF).

56. A pharmaceutical composition comprising the active compound cyclo(-Thr-Trp-Ile-Dab-Orn-^{D}Dab-Dab-Trp-Dab-Dab-Ala-Ser-^{D}Pro-Pro-);
or any pharmaceutically acceptable salt thereof;
and optionally one or more pharmaceutically acceptable diluents, excipients or carriers; for use in a method for the prevention, management or treatment of biofilm-associated diseases or conditions of the lungs in a subject.

57. A pharmaceutical composition comprising the active compound cyclo(-Thr-Trp-Ile-Dab-Orn-^{D}Dab-Dab-Trp-Dab-Dab-Ala-Ser-^{D}Pro-Pro-);
or any pharmaceutically acceptable salt thereof;
and optionally one or more pharmaceutically acceptable diluents, excipients or carriers; for use in a method for eradicating a biofilm in a biofilm-associated diseases or conditions of the lungs in a subject.

58. A pharmaceutical composition for use according to any one of claim 56 or 57, wherein the biofilm-associated diseases or conditions of the lungs are selected from the group consisting of cystic fibrosis (CF), non-cystic fibrosis bronchiectasis (NCFB), asthma, chronic obstructive pulmonary disease (COPD), ventilator-associated pneumonia (VAP), ventilator-associated bacterial pneumonia (VABP), hospital-acquired pneumonia (HAP), hospital-acquired bacterial pneumonia (HABP), and healthcare-associated pneumonia (HCAP).

59. A pharmaceutical composition for use according to any one of claim 56 or 57, wherein the biofilm-associated diseases or conditions of the lungs are cystic fibrosis (CF), or non-cystic fibrosis bronchiectasis (NCFB).

60. A pharmaceutical composition for use according to any one of claim 56 or 57, wherein the biofilm-associated disease or condition of the lungs is cystic fibrosis (CF).

61. A pharmaceutical composition comprising the active compound cyclo(-Thr-Trp-Ile-Dab-Orn-^{D}Dab-Dab-Trp-Dab-Dab-Ala-Ser-^{D}Pro-Pro-);
or any pharmaceutically acceptable salt thereof;
and optionally one or more pharmaceutically acceptable diluents, excipients or carriers; for use in a method for eradicating a biofilm in biofilm-associated diseases or conditions in a subject.

62. A pharmaceutical composition comprising the active compound cyclo(-Thr-Trp-Ile-Dab-Orn-^{D}Dab-Dab-Trp-Dab-Dab-Ala-Ser-^{D}Pro-Pro-);
or any pharmaceutically acceptable salt thereof;
and optionally one or more pharmaceutically acceptable diluents, excipients or carriers; for use in a method for the prevention, management or treatment of biofilm formation in a subject.
